(19) United States
(12) Patent Application Publication
(10) Pub. No.: US 2023/0029259 A1
(43) Pub. Date: Jan. 26, 2023

(54) COMBINATION OF IAP INHIBITORS AND PARP OR MEK INHIBITORS OR OTHER CHEMOTHERAPEUTIC AGENTS

(71) Applicants: Ascentage Pharma (Suzhou) Co., Ltd., Suzhou (CN); Ascentage Pharma Group Corp Limited, Hong Kong (CN)

(72) Inventors: Yifan Zhai, Suzhou (CN); Dajun Yang, Suzhou (CN); Douglas Dong Fang, Suzhou (CN); Qiang Li, Suzhou (CN)

(21) Appl. No.: 17/781,699

(22) PCT Filed: Dec. 2, 2020
(86) PCT No.: PCT/CN2020/133246

§ 371 Date: Jun. 1, 2022

(30) Foreign Application Priority Data
Dec. 2, 2019 (CN) PCT/CN2019/122402
Nov. 18, 2020 (CN) 202011295946.7

(51) Int. Cl. C07D 519/00; A61P 35/00

(52) U.S. Cl. CPC C07D 519/00; A61P 35/00

(57) ABSTRACT

A pharmaceutical composition comprising: a) an effective amount of an Inhibitors of Apoptosis Proteins (IAP) inhibitor, wherein the IAP inhibitor is represented by formula (I): or a pharmaceutically acceptable salt thereof, the definitions of each variable are provided herein; b) an effective amount of a second inhibitor, wherein the second inhibitor is a poly ADP ribose polymerase (PARP) inhibitor or a mitogen-activated protein kinase kinase (MEK) inhibitor; and a pharmaceutically acceptable carrier or diluent.

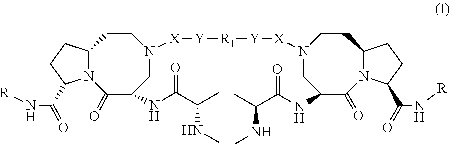

### FIELD OF THE INVENTION

The present invention relates to combination therapies of bivalent inhibitors of Inhibitors of Apoptosis Proteins (IAPs) and poly ADP ribose polymerase (PARP) inhibitors or mitogen-activated protein kinase kinase (MEK) inhibitors or other chemotherapeutic agents to treat cancers.

### BACKGROUND OF THE INVENTION

Apoptosis, or programmed cell death, is a cell process critical for homeostasis, normal development, host defense, and suppression of oncogenesis. Faulty regulation of apoptosis has been implicated in many human diseases,⁽¹⁾ including cancer,^{(1),(3)} and it is now recognized that resistance to apoptosis is a hallmark of cancer.⁽⁴⁾ As a consequence, targeting of key apoptosis regulators has emerged as an attractive strategy for the development of new approaches to human cancer treatment.⁽¹⁾

Most current cancer therapies, including chemotherapeutic agents, radiation, and immunotherapy, indirectly induce apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Such primary or acquired resistance of human cancers to current therapies due to apoptosis defects is a major problem in current cancer therapy.

In order to improve survival and quality of life of cancer patients, current and future efforts in the design and development of new molecular target-specific anticancer therapies includes strategies that specifically target cancer cell resistance to apoptosis. In this regard, targeting negative regulators that play a central role in directly inhibiting apoptosis in cancer cells represents a highly promising therapeutic strategy for new anticancer drug design.

One class of central negative regulators of apoptosis is the Inhibitors of Apoptosis Proteins (IAPs). This class includes proteins such as XIAP, cIAP1, cIAP2, ML-IAP, HIAP, KIAP, TSIAP, NAIP, survivin, livin, ILP-2, apollon, and BRUCE. IAP proteins potently suppress cancer cell apoptosis induced by a large variety of apoptotic stimuli, including chemotherapeutic agents, radiation, and immunotherapy.

Although their roles are not limited to regulation of apoptosis,^{(7),(8)} IAP proteins are a class of key apoptosis regulators, and are characterized by the presence of one or more BIR (Baculoviral IAP Repeat) domains.⁽⁵⁾⁻⁽⁶⁾ Among the IAPs, cellular IAP1 (cIAP1) and cIAP2 play a key role in the regulation of death-receptor mediated apoptosis, whereas X-linked IAP (XIAP) inhibits both death-receptor mediated and mitochondria mediated apoptosis by binding to and inhibiting caspase-3/7 and caspase-9, three cysteine proteases critical for execution of apoptosis.⁽⁵⁾ These IAP proteins are highly overexpressed both in cancer cell lines and in human tumor tissues and have low expression in normal cells and tissues.⁽⁹⁾ Extensive studies have demonstrated that overexpression of IAP proteins make cancer cells resistant to apoptosis induction by a variety of anticancer drugs.⁽¹⁰⁾⁻⁽¹²⁾ A detailed discussion of IAP proteins and their role is cancer and apoptosis is set forth in U.S. Pat. No. 7,960,372, incorporated herein by reference. Hence, targeting one or more of these IAP proteins is a promising therapeutic strategy for the treatment of human cancer.⁽¹⁰⁾⁻⁽¹²⁾ Studies have shown that peptide-based inhibitors are useful tools to elucidate the anti-apoptotic function of IAPs and the role of IAPs in the response of cancer cells to chemotherapeutic agents. However, peptide-based inhibitors have intrinsic limitations as useful therapeutic agents, including a poor cell permeability and poor in vivo stability. In published studies using Smac-based peptide inhibitors, the peptides had to be fused to carrier peptides to make them relatively cell-permeable.

Small molecule inhibitors of IAP proteins also are known. For example, International Patent Publication Application No. WO2014/031487 discloses dimeric Smac mimetic compounds.

In spite of numerous treatment options for patients with cancers, there remains a need for effective and safe molecularly targeted anti-cancer agents. Combination of such exploratory agents with existing therapies sometimes results in a synergistic interaction and enhanced therapeutic benefit relative to either agent alone.

### SUMMARY OF THE INVENTION

The present invention is based on the discovery that the administration of an IAP inhibitor or a pharmaceutically acceptable salt thereof and a PARP inhibitor or a MEK inhibitor or other chemotherapeutic agents synergistically treats a disease (e.g., a cancer). Specifically, as evidenced by Biological Examples 1 and 2 disclosed herein, it is surprising to find that the addition of an IAP inhibitor (e.g., the compound of Example 24) improves antitumor activity of a PARP inhibitor (e.g., olaparib), a MEK inhibitor (e.g., trametinib), or other chemotherapeutic agents (e.g., Abraxane and Gemcitabine).

Accordingly, in one aspect, the present disclosure provides a pharmaceutical composition comprising: a) an effective amount of an Inhibitors of Apoptosis Proteins (IAP) inhibitor, wherein the IAP inhibitor is represented by formula (I):

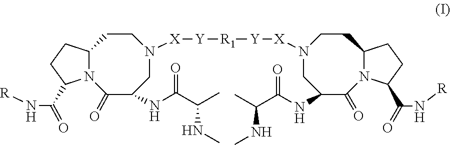

or a pharmaceutically acceptable salt thereof, the definitions of each variable are provided herein; b) an effective amount of a second inhibitor, wherein the second inhibitor is a poly ADP ribose polymerase (PARP) inhibitor or a mitogen-activated protein kinase kinase (MEK) inhibitor; and c) a pharmaceutically acceptable carrier or diluent.

In another aspect, the present disclosure provides a method of treating a disease (e.g., a cancer), comprising administering to a subject in need thereof: a) an effective amount of an Inhibitors of Apoptosis Proteins (IAP) inhibitor, wherein the IAP inhibitor is represented by formula (I):

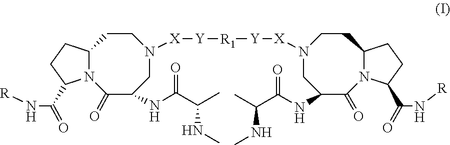

or a pharmaceutically acceptable salt thereof, the definitions of each variable are provided herein; b) an effective amount of a second inhibitor, wherein the second inhibitor is a poly ADP ribose polymerase (PARP) inhibitor or a mitogen-activated protein kinase kinase (MEK) inhibitor.

The disease can be treated by the method of the present disclosure includes a cancer, a T and B cell mediated autoimmune disease, and inflammatory disease; an infection, a hyperproliferative diseases, AIDS, a degenerative condition, or a vascular disease.

In another aspect, the present disclosure provides use of an IAP inhibitor represented by formula (I) (e.g., the compound of Example 24) in the preparation of a medicament for use in combination with a PARP inhibitor (e.g., olaparib) or a MEK inhibitor (e.g., trametinib) to treat a disease (e.g. a cancer).

In another aspect, the present disclosure provides an IAP inhibitor represented by formula (I) (e.g., the compound of Example 24) for use in combination with a PARP inhibitor (e.g., olaparib) or a MEK inhibitor (e.g., trametinib) to treat a disease (e.g. a cancer).

In still another aspect, the present disclosure provides a kit for human pharmaceutical use comprising (a) a container, (b1) a packaged composition comprising an IAP protein inhibitor represented by formula (I), and, (b2) a packaged composition comprising a second inhibitor selected from a PARP inhibitor (e.g., olaparib) or a MEK inhibitor (e.g., trametinib) useful in the treatment of a disease, and (c) a package insert containing directions for use of the composition or compositions, administered simultaneously or sequentially, in the treatment of the disease.

An IAP protein inhibitor represented by formula (I) and the second inhibitor can be administered together as a single-unit dose or separately as multi-unit doses, wherein the IAP inhibitor of structural formula (I) is administered before the second inhibitor or vice versa. It is envisioned that one or more dose of an IAP inhibitor of structural formula (I) and/or one or more dose of a second inhibitor can be administered.

In some embodiments, an IAP protein inhibitor of structural formula (I) and a second inhibitor are administered simultaneously. In some embodiments, the IAP protein inhibitor of structural formula (I) and second inhibitor are administered from a single composition or from separate compositions. In some embodiments, the IAP protein inhibitor of structural formula (I) and second inhibitor are administered sequentially. An IAP protein inhibitor of structural formula (I), as used in the present invention, can be administered in an amount of about 0.005 to about 500 milligrams per dose, about 0.05 to about 250 milligrams per dose, or about 0.5 to about 100 milligrams per dose.

### BRIEF DESCRIPTION OF THE DRAWING

FIG. 1 shows antitumor activity of the compound of Example 24 in combination with olaparib or trametinib in the treatment of pancreatic cancer patient derived xenograft (PDX) model PA1170 (Mean±SEM) in Biological Example 1.

FIG. 2 shows body weight change (%) of mice bearing PA1170 pancreatic cancer patient derived xenograft tumors under the treatment with the compound of Example 24, olaparib and trametinib (Mean±SEM) in Biological Example 1.

FIG. 3 shows antitumor activity of the compound of Example 24 in combination with olaparib or trametinib in the treatment of pancreatic cancer patient derived xenograft model PA6265 (Mean±SEM) in Biological Example 1.

FIG. 4 shows body weight change (%) of mice bearing PA6265 pancreatic cancer patient derived xenograft tumors under the treatment with the compound of Example 24, trametinib and olaparib (Mean±SEM) in Biological Example 1.

FIG. 5 shows antitumor activity of the compound of Example 24 in combination with abraxane plus gemcitabine in the treatment of pancreatic cancer patient derived xenograft model PA0787 (Mean±SEM) in Biological Example 1.

FIG. 6 shows body weight change (%) of mice bearing PA0787 pancreatic cancer patient derived xenograft tumors under the treatment with the compound of Example 24 and abraxane plus gemcitabine (Mean±SEM) in Biological Example 1.

FIG. 7 shows antitumor activity of the compound of Example 24 in combination with abraxane plus gemcitabine in the treatment of pancreatic cancer patient derived xenograft model PA1194 (Mean±SEM) in Biological Example 1.

FIG. 8 shows body weight change (%) of mice bearing PA1194 pancreatic cancer patient derived xenograft tumors under the treatment with the compound of Example 24 and abraxane plus gemcitabine (Mean±SEM) in Biological Example 1.

FIG. 9 shows antitumor activity of the compound of Example 24 in combination with abraxane plus gemcitabine in the treatment of murine pancreatic cancer xenograft model mPA6115 (Mean±SEM) in Biological Example 2.

FIG. 10 shows body weight change (%) of mice bearing mPA6115 murine pancreatic tumors under the treatment with the compound of Example 24 and abraxane plus gemcitabine (Mean±SEM) in Biological Example 2.

FIG. 11 shows antitumor activity of compound of EXAMPLE 24 in combination with olaparib and trametinib in the treatment of pancreatic cancer patient derived xenograft model PA1170 (Mean±SEM) in embodiment 1.

FIG. 12 shows body weight change (%) of mice bearing PA1170 pancreatic cancer patient derived xenograft tumors under the treatment with compound of EXAMPLE 24, olaparib and trametinib (Mean±SEM) in embodiment 1.

FIG. 13 shows antitumor activity of compound of EXAMPLE 24 in combination with abraxane plus gemcitabine in the treatment of Panc-1 human pancreatic cancer xenograft model in mice.

FIG. 14 shows body weight change (%) of mice bearing Panc-1 human pancreatic cancer xenograft tumor under the treatment with compound of EXAMPLE 24 and abraxane plus gemcitabine (Mean±SEM).

### DETAILED DESCRIPTION OF THE INVENTION

### Definitions

Unless otherwise defined hereinafter, all technical and scientific terms used herein are intended to have the same meaning as commonly understood by those skilled in the art. Reference to techniques used herein is intended to refer to techniques commonly understood in the art, including those that are obvious to those skilled in the art as variations of techniques or substitutions of equivalent techniques. While the following terms are believed to be well understood by those skilled in the art, the following definitions are set forth to better explain the present disclosure.

As used herein, the terms “including”, “comprising”, “having”, “containing”, or “involving” and other variations thereof are inclusive or open-ended herein, and do not exclude other unlisted elements or method steps.

As used herein, the term “alkyl” refers to straight chained and branched saturated C₁₋₁₀ hydrocarbon groups, nonlimiting examples of which include methyl, ethyl, and straight chain and branched propyl, butyl, pentyl, hexyl, heptyl, octyl, nonyl, and decyl groups. The term Cₙ means the alkyl group has “n” carbon atoms.

The term “C₃₋₆cycloalkylene” refers to a disubstituted cycloalkane having 3 to 6 carbon atoms, for example

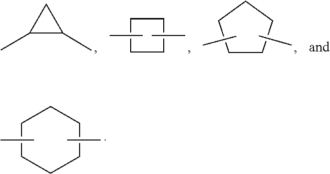

The “C₃₋₆cycloalkylene” can be unsubstituted, or substituted with 1 to 3 groups, for example, C₁₋₄alkyl, halo, trifluoromethyl, trifluoromethoxy, hydroxy, alkoxy, nitro, cyano, alkylamino, or amino groups.

The term “alkenyl” is defined identically as “alkyl,” except for containing a carbon-carbon double bond, e.g., ethenyl, propenyl, and butenyl.

As used herein, the term “halo” is defined as fluoro, chloro, bromo, and iodo.

The term “hydroxy” is defined as —OH.

The term “alkoxy” is defined as —OR, wherein R is alkyl.

The term “amino” is defined as —NH₂, and the term “alkylamino” is defined as —NR₂, wherein at least one R is alkyl and the second R is alkyl or hydrogen.

The term “nitro” is defined as —NO₂.

The term “cyano” is defined as —CN.

The term “trifluoromethyl” is defined as —CF₃.

The term “trifluoromethoxy” is defined as —OCF₃.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four, groups independently selected from, for example, halo, alkyl, alkenyl, —OCF₃, —NO₂, —CN, —NC, —OH, alkoxy, amino, alkylamino, —CO₂H, —CO₂alkyl, alkynyl, cycloalkyl, nitro, sulfhydryl, imino, amido, phosphonate, phosphinate, silyl, alkylthio, sulfonyl, sulfonamide, aldehyde, heterocycloalkyl, trifluoromethyl, aryl, and heteroaryl.

As used herein, the term “heteroaryl” refers to a monocyclic or bicyclic ring system containing one or two aromatic rings and containing at least one and up to four nitrogen atoms in an aromatic ring. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, alkylamino, —CO₂H, —CO₂alkyl, alkynyl, cycloalkyl, nitro, sulfhydryl, imino, amido, phosphonate, phosphinate, silyl, alkylthio, sulfonyl, sulfonamide, aldehyde, heterocycloalkyl, trifluoromethyl, aryl, and heteroaryl.

The term “arylene” refers to a bidentate aryl group that bonds to two other groups and serves to connect these groups, e.g.,

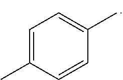

The term “heteroarylene” is similarly defined.

Nonlimiting examples of aryl groups are

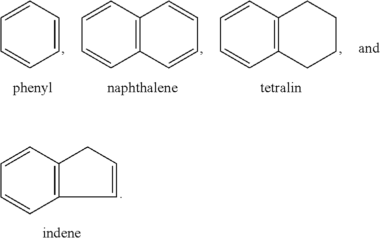

Nonlimiting examples of heteroaryl groups are

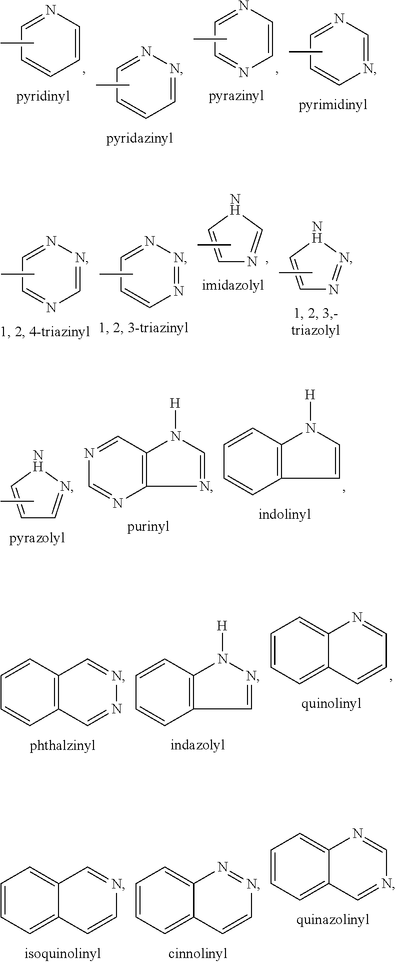

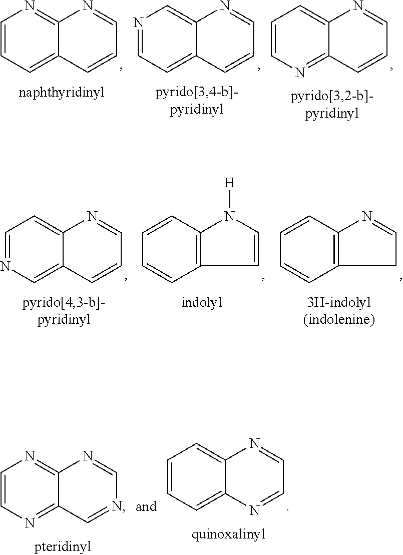

The term “IAP proteins,” as used herein, refers to any known member of the Inhibitors of Apoptosis Protein family, including, but not limited to, XIAP, cIAP-1, cIAP-2, ML-IAP, HIAP, TSIAP, KIAP, NAIP, survivin, livin, ILP-2, apollon, and BRUCE.

The term “overexpression of IAPs,” as used herein, refers to an elevated level (e.g., aberrant level) of mRNAs encoding for an IAP protein(s), and/or to elevated levels of IAP protein(s) in cells as compared to similar corresponding non-pathological cells expressing basal levels of mRNAs encoding IAP proteins or having basal levels of IAP proteins. Methods for detecting the levels of mRNAs encoding IAP proteins or levels of IAP proteins in a cell include, but are not limited to, Western blotting using IAP protein antibodies, immunohistochemical methods, and methods of nucleic acid amplification or direct RNA detection. As important as the absolute level of IAP proteins in cells is to determining that they overexpress IAP proteins, so also is the relative level of IAP proteins to other pro-apoptotic signaling molecules (e.g., pro-apoptotic Bcl-2 family proteins) within such cells. When the balance of these two are such that, were it not for the levels of the IAP proteins, the pro-apoptotic signaling molecules would be sufficient to cause the cells to execute the apoptosis program and die, said cells would be dependent on the IAP proteins for their survival. In such cells, exposure to an inhibiting effective amount of an IAP protein inhibitor will be sufficient to cause the cells to execute the apoptosis program and die. Thus, the term “overexpression of an IAP protein” also refers to cells that, due to the relative levels of pro-apoptotic signals and anti-apoptotic signals, undergo apoptosis in response to inhibiting effective amounts of compounds that inhibit the function of IAP proteins.

The term “a disease or condition wherein inhibition of an IAP protein provides a benefit” pertains to a condition in which an IAP protein, and/or an action of an IAP protein, is important or necessary, e.g., for the onset, progress, expression of that disease or condition, or a disease or a condition which is known to be treated by an IAP protein inhibitor. An example of such a condition includes, but is not limited to, a cancer. One of ordinary skill in the art is readily able to determine whether a compound treats a disease or condition mediated by an IAP protein for any particular cell type, for example, by assays which conveniently can be used to assess the activity of particular compounds.

The term “disease” or “condition” denotes disturbances and/or anomalies that as a rule are regarded as being pathological conditions or functions, and that can manifest themselves in the form of particular signs, symptoms, and/or malfunctions. A compound of structural formula (I) disclosed herein is a potent inhibitor of IAP proteins and can be used in treating diseases and conditions wherein inhibition an IAP protein provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a compound of the invention to an individual in need of such treatment.

The terms “sensitize” and “sensitizing,” as used herein, refer to making, through the administration of a first agent (e.g., a compound of structural formula I), an animal or a cell within an animal more susceptible, or more responsive, to the biological effects (e.g., promotion or retardation of an aspect of cellular function including, but not limited to, cell division, cell growth, proliferation, invasion, angiogenesis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least 10%, at least 20%, at least 30%, at least 40%, at least 50%, at least 60%, at least 70%, at least 80%, at least 90%, at least 100%, at least 150%, at least 200%, at least 350%, at least 300%, at least 350%, at least 400%, at least 450%, or at least 500% over the response in the absence of the first agent.

The term “hyperproliferative disease,” as used herein, refers to any condition in which a localized population of proliferating cells in an animal is not governed by the usual limitations of normal growth. Examples of hyperproliferative disorders include, but are not restricted to tumors, neoplasms, lymphomas and the like. A neoplasm is said to be benign if it does not undergo invasion or metastasis, and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells, or tissues. Nonlimiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “dysregulation of apoptosis,” as used herein, refers to any aberration in the ability of (e.g., predisposition) a cell to undergo cell death via apoptosis. Dysregulation of apoptosis is associated with or induced by a variety of conditions, including for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis. It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

The term “therapeutically effective amount”, “effective amount”, or “effective dose” as used herein refers to an amount of the active ingredient(s) that is(are) sufficient, when administered by a method of the present disclosure, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; reduce IAP protein signaling in the target cells increase survival time; and/or relieve, to some extent, one or more of the symptoms associated with the cancer by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. To the extent the administered compound or composition prevents growth and/or kills existing cancer cells, it may be cytostatic and/or cytotoxic. A therapeutically effective amount can be given in unit dosage form (e.g., 0.1 mg to about 50 g per day, alternatively from 1 mg to about 5 grams per day; and in another alternatively from 10 mg to 1 gram per day).

The term “container” means any receptacle and closure therefor suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, an IAP protein inhibitor of structural formula (I) can be administered at the same time or sequentially in any order at different points in time as a second inhibitor. An IAP protein inhibitor of structural formula (I) and the second inhibitor can be administered separately or sequentially, in any appropriate form and by any suitable route. When a present IAP protein inhibitor and the second inhibitor are not administered concurrently, it is understood that they can be administered in any order to a subject in need thereof. For example, a present IAP protein inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second inhibitor, to an individual in need thereof. In various embodiments, an IAP protein inhibitor of structural formula (I) and the second inhibitor are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at 1 minute to 24 hours apart.

The use of the terms “a”, “an”, “the”, and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

### IAP Inhibitors

The compounds of structural formula (I) disclosed herein are mimetics of Smac and function as inhibitors of IAPs proteins. The compounds sensitize cells to inducers of apoptosis and, in some instances, themselves induce apoptosis by inhibiting IAPs proteins. Therefore, the present disclosure provides methods of sensitizing cells to inducers of apoptosis, and to methods of inducing apoptosis in cells, comprising contacting the cells with a compound of structural formula (I) alone or in combination with an inducer of apoptosis (e.g., a second inhibitor selected from a PARP inhibitor or a MEK inhibitor). The present disclosure further provides methods of treating or ameliorating disorders in an animal that are responsive to induction of apoptosis comprising administering to the animal a compound of structural formula (I) and an inducer of apoptosis (e.g., a second inhibitor selected from a PARP inhibitor or a MEK inhibitor). Such disorders include those characterized by a dysregulation of apoptosis and those characterized by overexpression of IAP proteins.

In some embodiments, IAP protein inhibitors disclosed herein have a structural formula (I):

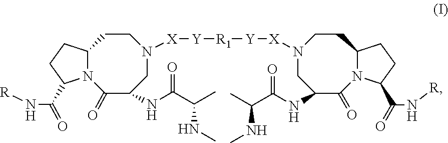

wherein X is selected from the group consisting of

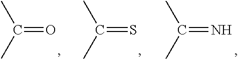

### and —SO₂—;

Y is selected from the group consisting of —NH—, —O—, —S—, and null;

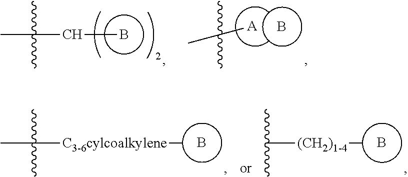

wherein ring A is an optionally substituted C₄₋₈ cycloalkyl (for example, optionally substituted with 1 to 3 groups selected from C₁₋₄alkyl, halo, trifluoromethyl, trifluoromethoxy, hydroxy, alkoxy, nitro, cyano, alkylamino, or amino groups), each ring B represented in R is independently optionally substituted aryl or optionally substituted nitrogen atom-containing heteroaryl; and

R₁ is selected from the group consisting of —(CH₂)₄₋₁₀—,

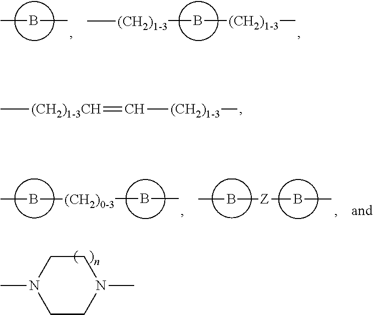

wherein Z is O, S, or NH; n is 0, 1, or 2, and each ring B represented in R₁ is independently optionally substituted aryl or optionally substituted nitrogen atom-containing heteroaryl; or a pharmaceutically acceptable salt, hydrate, solvate, or prodrug thereof.

In one embodiment, each ring B represented in R is independently optionally substituted with halo, CF₃, or both.

In one embodiment, each ring B represented in R₁ is independently optionally substituted with C₁₋₃ alkyl.

The compounds of structural formula (I) inhibit IAP proteins and are useful in the treatment of a variety of diseases and conditions. In particular, the compounds of structural formula (I) are used in methods of treating a disease or condition wherein inhibition of an IAP protein provides a benefit, for example, cancers, autoimmune disorders, and chronic inflammatory conditions. The method comprises administering a therapeutically effective amount of a compound of structural formula (I) to an individual in need thereof. The present methods also encompass administering a second therapeutic agent to the individual in addition to the compound of structural formula (I). The second therapeutic agent is selected from drugs known as useful in treating the disease or condition afflicting the individual in need thereof, e.g., a chemotherapeutic agent and/or radiation known as useful in treating a particular cancer.

In some embodiments, each ring B is independently optionally substituted phenyl, optionally substituted naphthyl, optionally substituted pyridinyl, optionally substituted pyridazinyl, optionally substituted pyrazinyl, or optionally substituted pyrimidinyl.

In some embodiments, R is

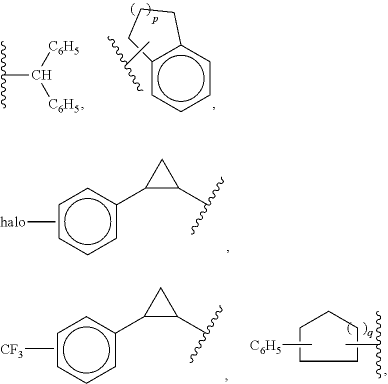

or —(CH₂)₂₋₄—C₆H₅;

p is 0 to 4, and q is 0 to 2.

In one embodiment, R is

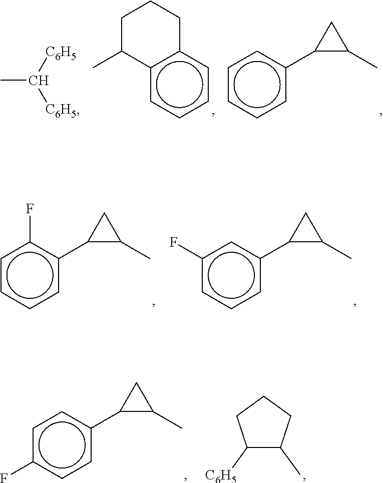

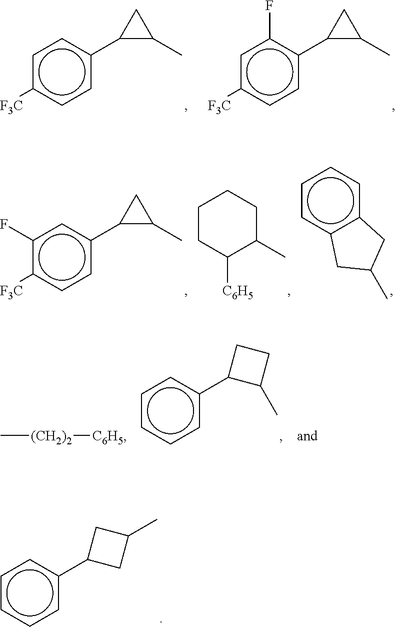

In some embodiments, R₁ is —(CH₂)₄₋₈—, —(CH₂)₄₋₈—,

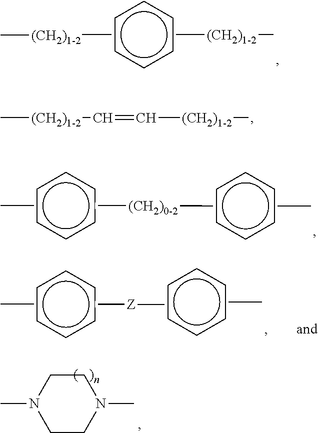

wherein n is 0 or 1.

In one embodiment, R₁ is —(CH₂)₄—, —(CH₂)₆—, —(CH₂)₈—,

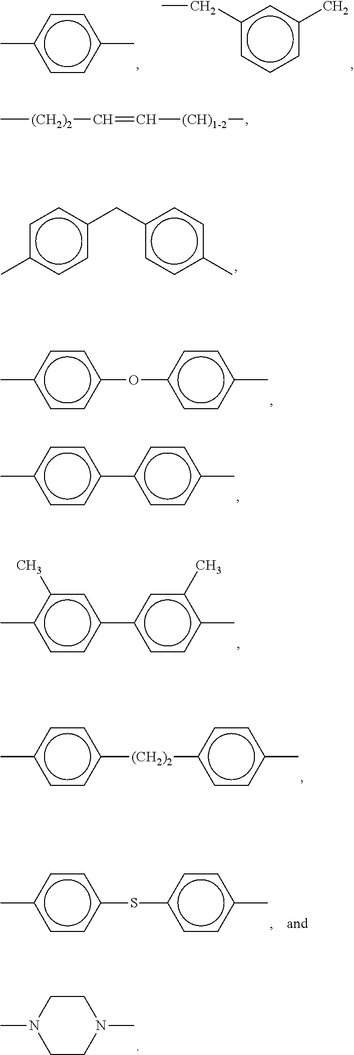

In some embodiments, X is

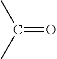

### and Y is —NH—.

In some embodiments, X is SO₂ and Y is null.

In some embodiments, X is

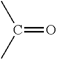

and Y is null.

In some embodiments, X is

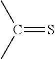

### and Y is —NH—.

In some embodiments, X is

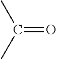

### and Y is —O—.

Additionally, salts, hydrates, solvates, and prodrugs of the compounds disclosed herein are also included and can be used in the methods of the present invention. The present invention further includes all possible stereoisomers and geometric isomers of the compounds of structural formula (I). The present invention includes both racemic compounds and optically active isomers. When a compound of structural formula (I) is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the compounds of structural formula (I) are possible, the present invention is intended to include all tautomeric forms of the compounds.

The compounds disclosed herein can exist as salts. Pharmaceutically acceptable salts of the compounds of the invention often are preferred in the methods of the invention. The term “pharmaceutically acceptable salt,” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. The term “pharmaceutically acceptable salts” also refers to zwitterionic forms of the compounds of structural formula (I). Salts of compounds of formula (I) can be prepared during the final isolation and purification of the compounds or separately by reacting the compound with an acid having a suitable cation. The pharmaceutically acceptable salts of compounds of structural formula (I) can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides.

Compounds of structural formula (I) can contain one or more asymmetric center, and therefore can exist as stereoisomers. The present invention includes both mixtures and individual stereoisomers. In particular, the compounds of structural formula (I) include both the individual cis- and trans-isomers, and mixtures of the cis- and trans-isomers, e.g.,

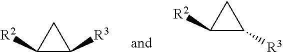

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. The term “geometric isomer” refers to compounds having at least one double bond, wherein the double bond(s) may exist in cis (also referred to as syn or entgegen (E)) or trans (also referred to as anti or zusammen (Z)) forms as well as mixtures thereof. When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geomeric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

The term “prodrug,” as used herein, refers to a pharmacologically inactive derivative of a parent “drug” molecule that requires biotransformation (e.g., either spontaneous or enzymatic) within the target physiological system to release, or to convert (e.g., enzymatically, physiologically, mechanically, electromagnetically) the prodrug into the active drug. Prodrugs are designed to overcome problems associated with stability, toxicity, lack of specificity, or limited bioavailability.

Prodrugs often offer advantages of solubility, tissue compatibility, or delayed release in the mammalian organism. (See e.g., Bundgard, “Design of Prodrugs”, pp. 7-9, 21-24, Elsevier, Amsterdam (1985); and Silverman, “The Organic Chemistry of Drug Design and Drug Action”, pp. 352-401, Academic Press, San Diego, Calif. (1992)). Exemplary prodrugs comprise an active drug molecule itself and a chemical masking group (e.g., a group that reversibly suppresses the activity of the drug). Some preferred prodrugs are variations or derivatives of compounds that have groups cleavable under metabolic conditions. Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Common prodrugs include acid derivatives such as esters prepared by reaction of parent acids with a suitable alcohol (e.g., a lower alkanol), amides prepared by reaction of the parent acid compound with an amine, or basic groups reacted to form an acylated base derivative (e.g., a lower alkylamide).

Specific compounds of structural formula (I) include, but are not limited to, compounds having the structure set forth below.

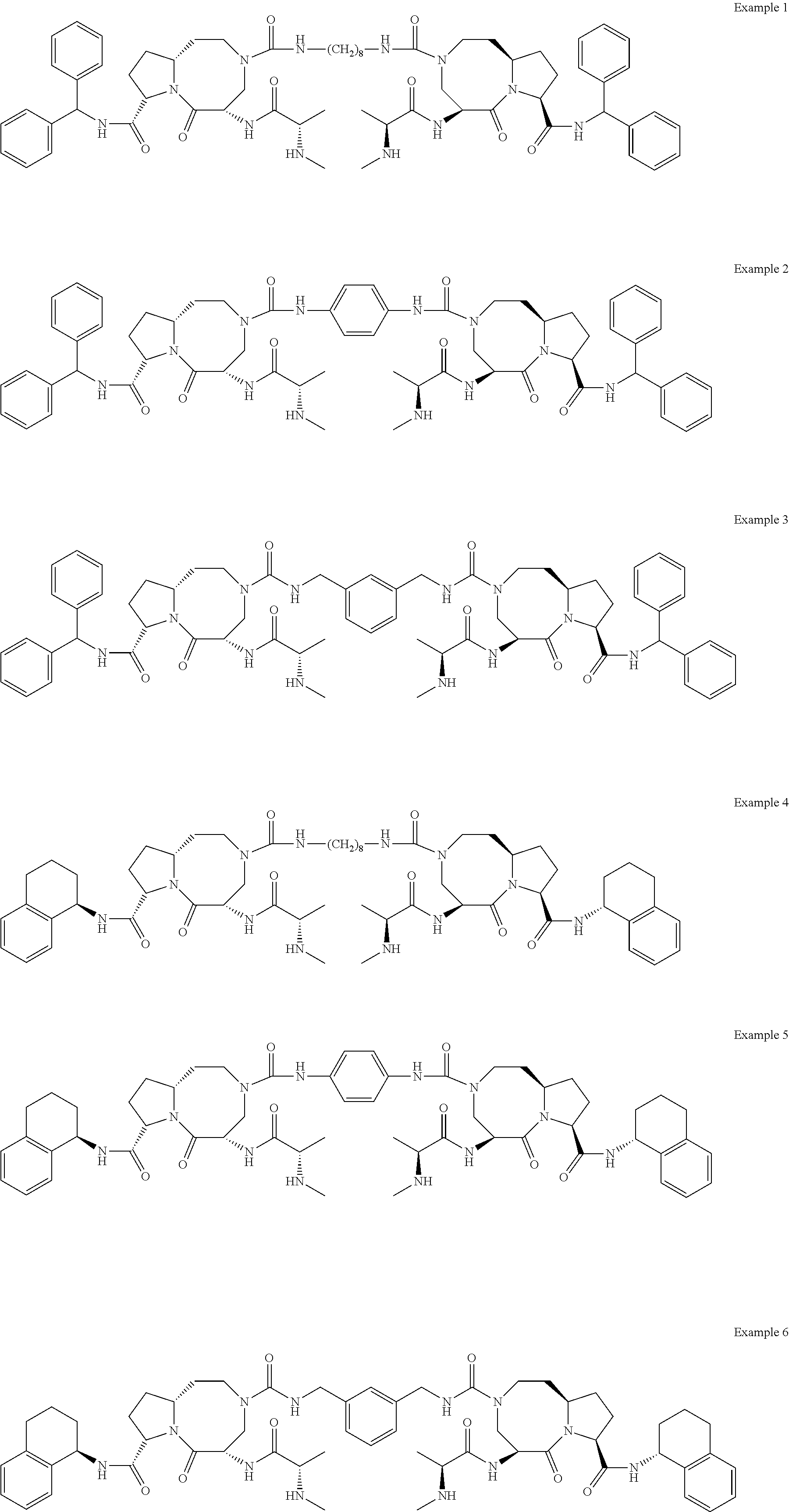

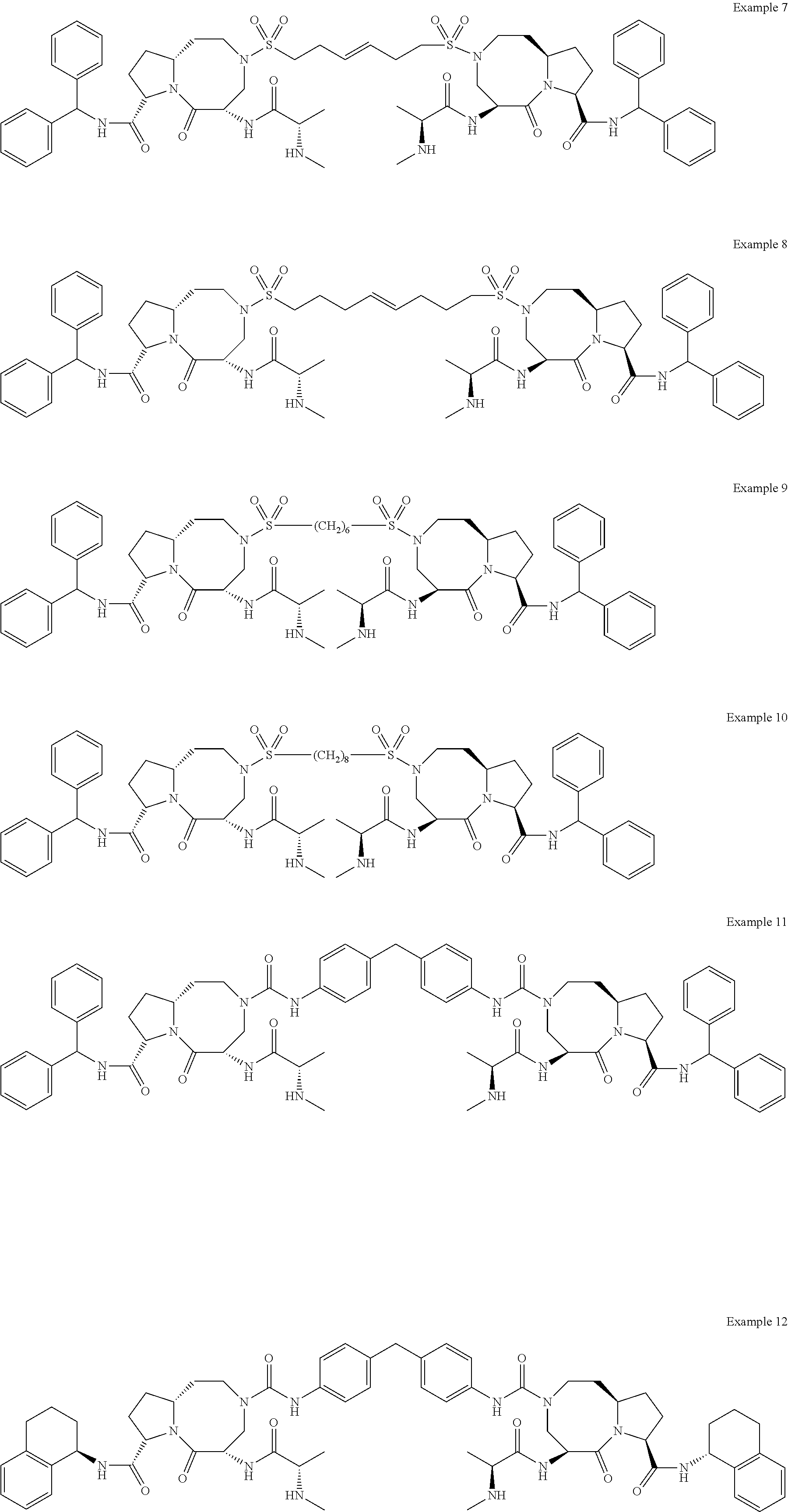

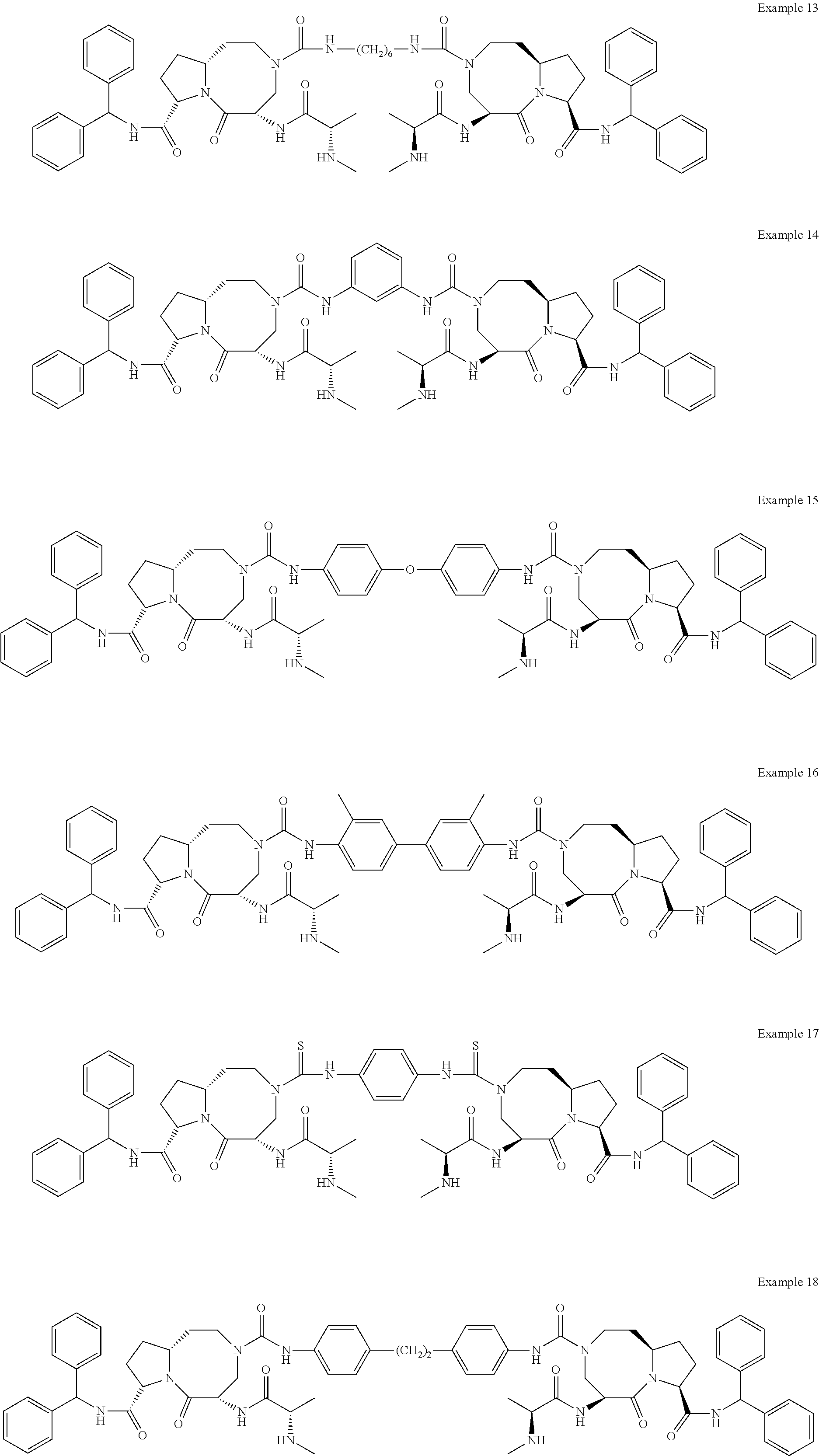

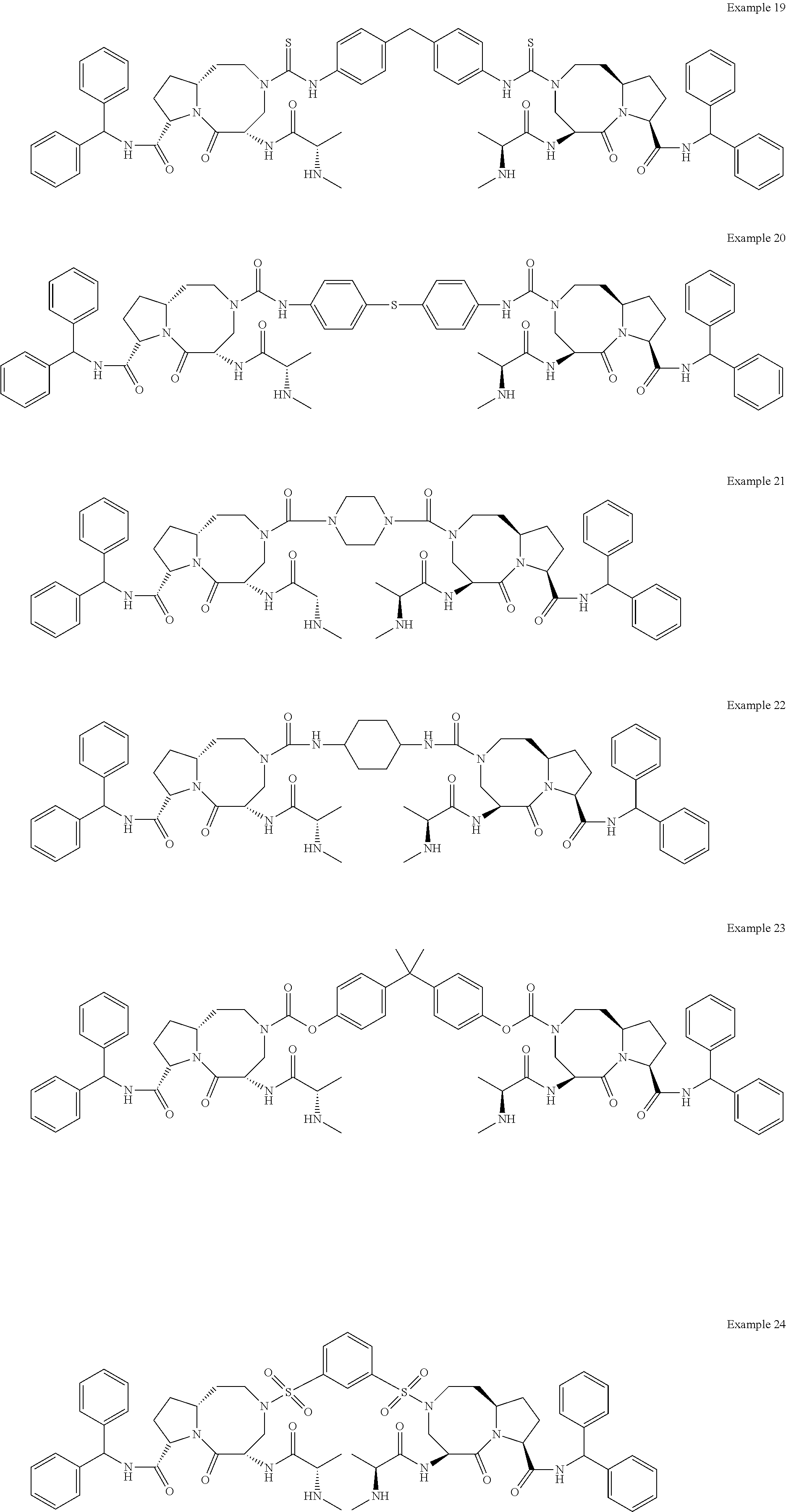

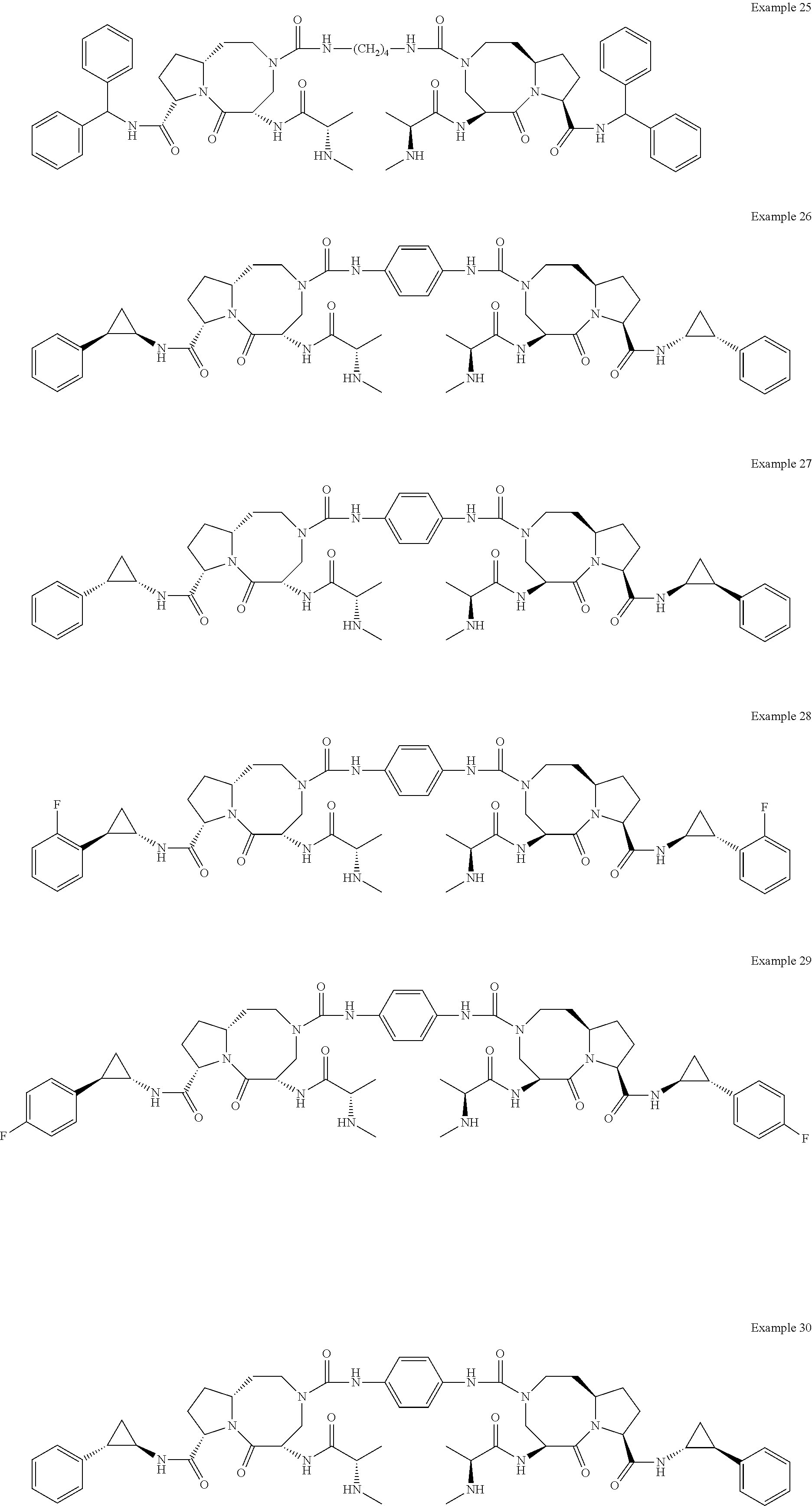

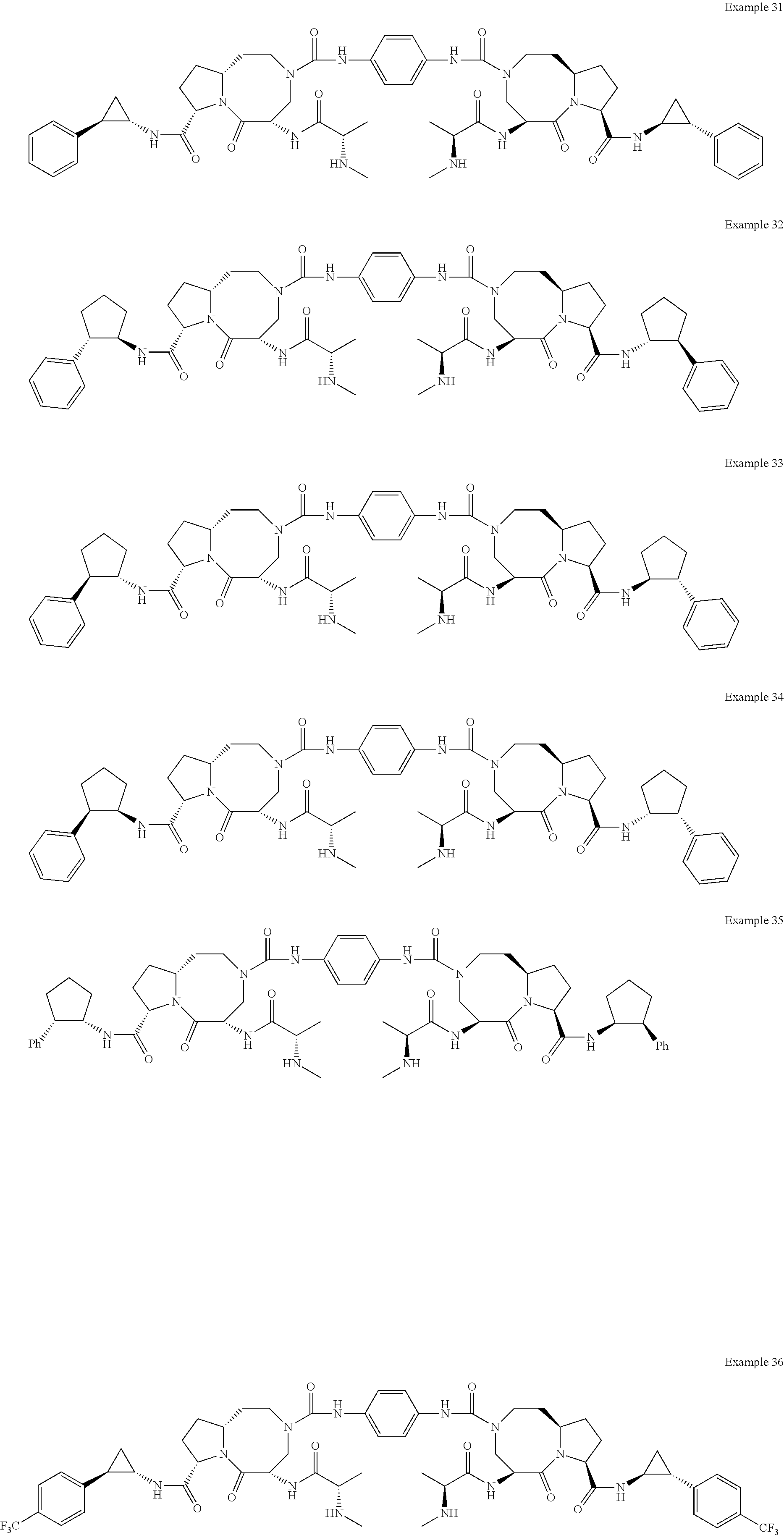

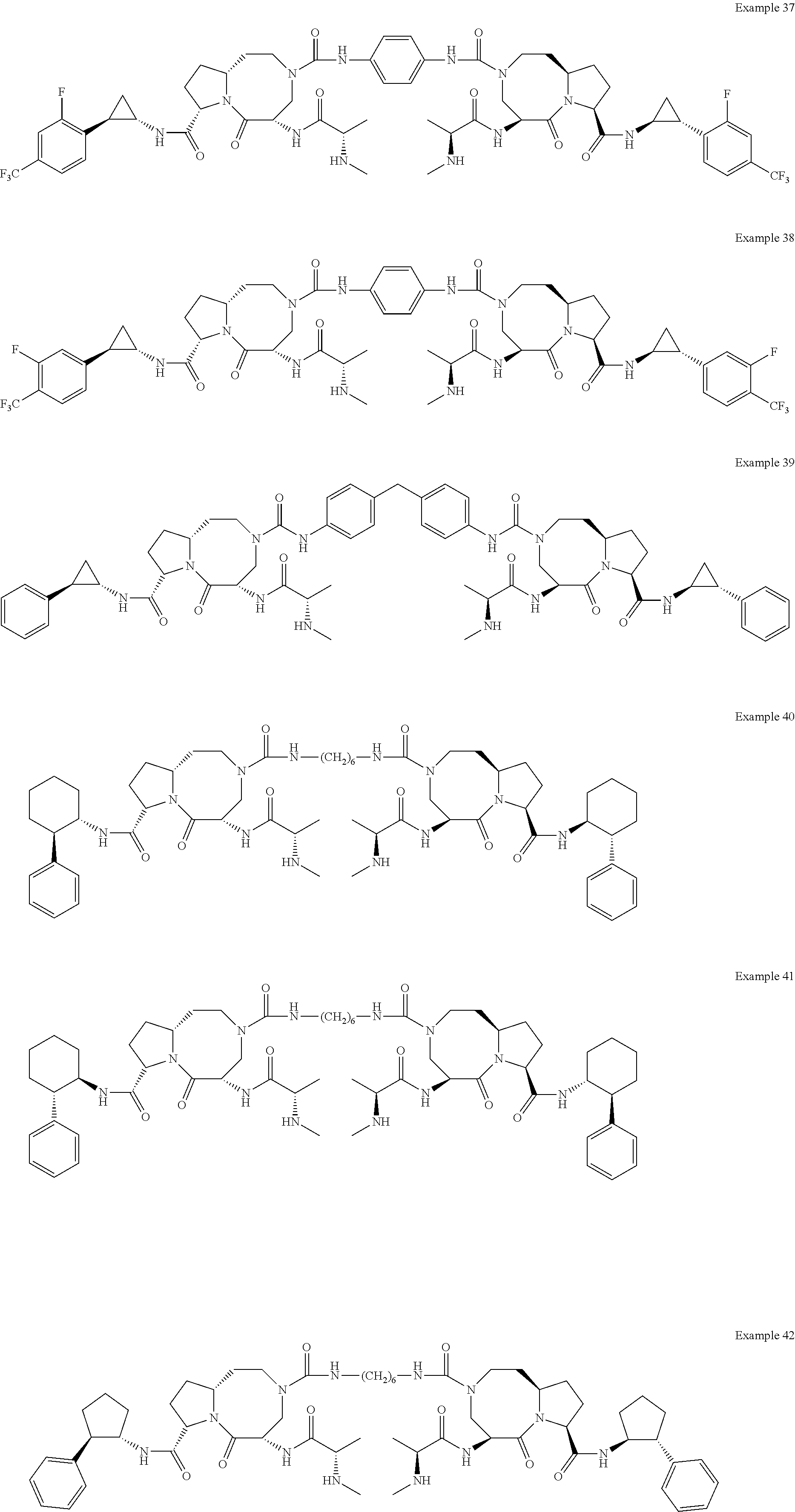

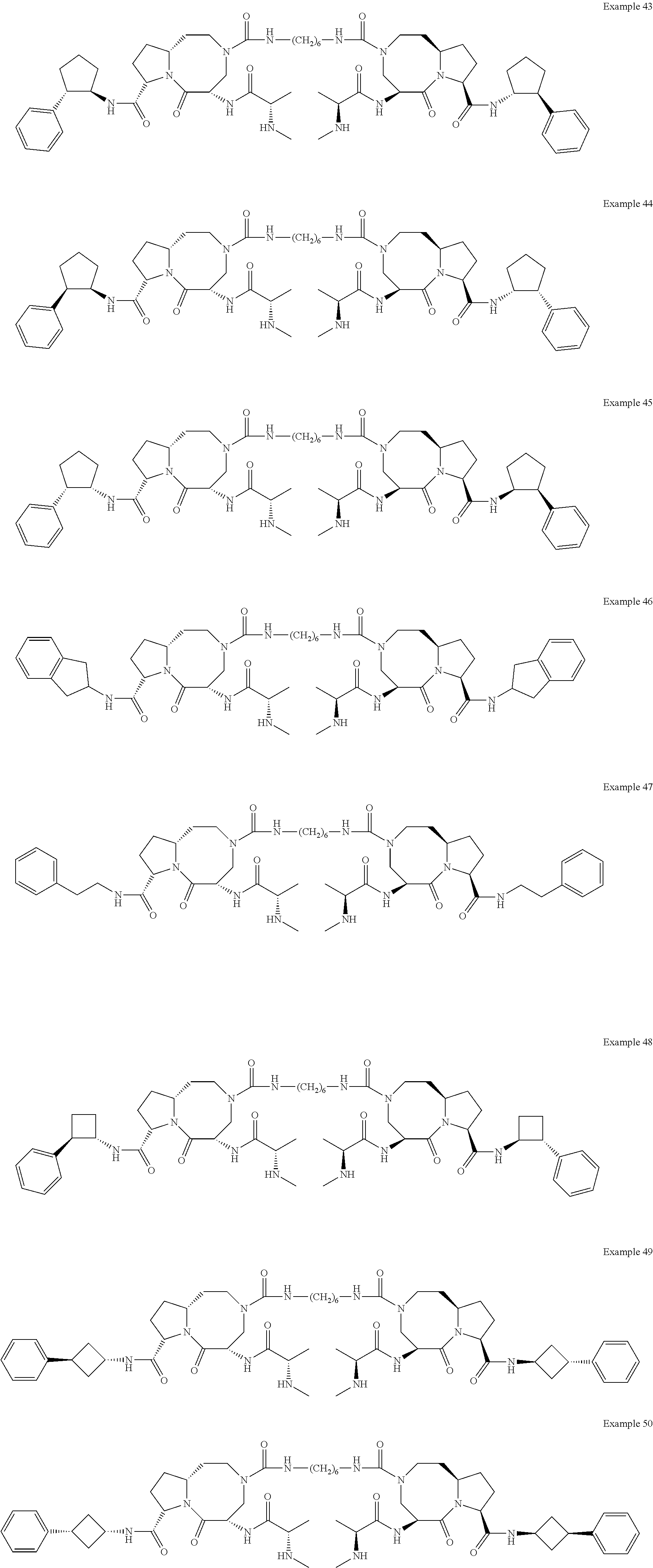

### PARP Inhibitors

The nuclear enzyme poly(ADP-ribose) polymerase-1 (PARP-1) is a member of the PARP enzyme family. This growing family of enzymes consists of PARPs such as, for example: PARP-1, PARP-2, PARP-3 and Vault-PARP.

PARP plays a role in the repair of DNA strand breaks and its inhibition is therefore an established approach to cancer treatment. PARP inhibition can be especially effective when combined with DNA damaging treatment, such as with ionizing radiation or after treatment with DNA damaging agents such as methylating agents, topoisomerases I inhibitors and other chemotherapeutic agents such as cisplatin and bleomycin. The inhibition of PARP enzymatic activity should lead to an enhanced sensitivity of the tumor cells towards DNA damaging treatments. PARP inhibitors have been reported to be effective in radiosensitizing (hypoxic) tumor cells and effective in preventing tumor cells from recovering from potentially lethal and sublethal damage of DNA after radiation therapy, presumably by their ability to prevent DNA strand break rejoining and by affecting several DNA damage signaling pathways.

The inhibition of PARP-2 can provide protection against oxidative stress (see Szanto, et al., Cell Mol. Life Sci. 69:4079 (2012)). As such, PARP inhibitors can be used to treat diseases characterized by oxidative stress (e.g., ischemia-reperfusion injury, inflammatory diseases, burn, Parkinsonism, Huntington's diseases, Alzheimer's disease and toxic insults).

PARP-1 and PARP-2 are pro-inflammatory (see Rosado et al., Immunology 139:428 (2013)). Their inhibition, as such, can be used to treat, for example, asthma, arthritis, colitis, chronic obstructive pulmonary disease (COPD), acute respiratory distress syndrome (ARDS), atherosclerosis, cardia remodeling after myocardial infarction, sepsis, endotoxic shock, hemorrhagic shock, graft-versus-host disease, encephalomyelitis and autoimmune nephritis.

PARP inhibition can also protect against viral infections (see Atasheva et al., J. Virol. 88:2116 (2014) and Virag and Szabo Pharmacol. Rev. 54:375 (2002)), e.g., against human immune deficiency virus 1, Venezuelan equine encephalitis virus, herpes simplex virus, human hepatitis B virus, and human cytomegalovirus infections (Virag and Szabo Pharmacol. Rev. 54:375 (2002)).

PARPs are involved in the control of glucose homeostasis (see Bai and Canto Cell Metab. 16:290 (2012), Riffel et al., Nat. Rev. Drug Discovery 11:923 (2012) and Yeh et al., Diabetes 58:2476 (2009). For example, PARP-1 inhibition improves glucose disposal and insulin sensitivity (see Bai and Canto Cell Metab. 16:290 (2012) and Pirinen et al., Cell Metab. 19:1034 (2014)). As such, PARP inhibition is useful for treating disease and conditions such as metabolic syndrome and type II diabetes and their subsequent complications such as diabetic neurological, renal and ocular complications.

PARPs are involved in a wide array of cellular functions, including DNA repair, mitochondrial homeostasis, protection against oxidative stress, inflammation, metabolic regulation, circadian rhythms, differentiation and aging. See, for example, Peter Bai, Molecular Cell 58:947 (2015). As such, PARP inhibitors have the potential to treat a wide range ailments, and a number of PARP inhibitors have been approved for the treatment of cancer.

The present disclosure includes, but is not limited to, the following PARP inhibitors: talazoparib, niraparib, rucaparib, olaparib, pamiparib, fluazolepali, veliparib, amelparib, CK-102, 2X-121, simmiparib, SC-10914, IMP-4297, ABT-767, MP-124, RBN-2397, IDX-1197, JPI-547, HWH-340, ZYTP-1, NT-125, AST-6828, OX-401, HC-X014, CBX-11, OC-301, TSL-1502, STP06-1002, mefuparib, R-554, AZ-6102, BPI-7000, SRX-3128, JNJ-928, AZD-2461, iniparib, ONO-2231, INO-1001, INO-1003, E-7016, LT-626, JPI-283, MK-2512, R-503, NMS-P914A, HYDAMTIQ, KR-33889, S-111, ANG-2864, PD-141703, PD-141076, PD-128763, BSI-401, A-620223, AAI-028, DR-2313, and BGP-15.

In some embodiments, the PARP inhibitor used in the present invention is olaparib, rucaparib, niraparib, talazoparib, veliparib, pamiparib, fluazolepali, amelparib, simmiparib, mefuparib, or iniparib. In one specific embodiment, the PARP inhibitor is olaparib.

### MEK Inhibitors

Mitogen-activated protein kinase kinase (also known as MAP2K, MEK, MAPKK) is a kinase enzyme which phosphorylates mitogen-activated protein kinase (MAPK).

Over activation of the mitogen-activated protein (MAP) kinase cascade is known to play an important role in cell proliferation and differentiation. This pathway can be activated when a growth factor binds to its receptor tyrosine kinase. This interaction promotes RAS association with RAF and initiates a phosphorylation cascade through MEK (MAP kinase) to ERK. The only known substrates for MEK phosphorylation are the MAP kinases, ERK1 and ERK2. Phosphorylation of MEK increases its affinity and catalytic activity toward ERK as well as its affinity for ATP. Constitutive activation of the MAPK pathway has been found in a number of diseases, for example, melanoma, pancreatic, colon, lung, kidney and ovarian cancers; in particular pancreatic, colon, lung, kidney and ovarian cancers. Therefore, inhibition of this pathway, particularly inhibiting MEK activity, is known to be beneficial in treating hyperproliferative diseases.

The present disclosure includes, but is not limited to, the following MEK inhibitors: binimetinib, cobimetinib, trametinib, LNP-3794, HL-085, antroquinonol, E-6201, refametinib, mirdametinib, pimasertib, selumetinib, SHR-7390, CKI-27, GS-4875, ATR-001, ATR-002, ATR-006, ATR-004, ATR-005, CS-3006, FCN-159, CIP-137401, EBI-1051, SC-1-151, SRX-2626, EDV-2209, WX-554, GDC-0623, TAK-733, E-6201, RG-7167, AZD-8330, PD-184352, GSK-2091976A, AS-703988, BI-847325, JTP-70902, CZ-775, R04987655, RO5126766, RO-5068760, RDEA-436, MEK-300, AD-GL0001, SL-327, CI-1040, CInQ-03, G-573, PD184161, PD318088, PD98059, U0126, and SL327.

In some embodiments, the MEK inhibitor is trametinib.

### Pharmaceutical Compositions

The present disclosure provides a pharmaceutical composition comprising a) an effective amount of an Inhibitors of Apoptosis Proteins (IAP) inhibitor, wherein the IAP inhibitor is represented by formula (I) or a pharmaceutically acceptable salt thereof as described above, b) an effective amount of a second inhibitor, wherein the second inhibitor is a poly ADP ribose polymerase (PARP) inhibitor (or a pharmaceutically acceptable salt thereof) or a mitogen-activated protein kinase kinase (MEK) inhibitor (or a pharmaceutically acceptable salt thereof) as disclosed herein; and c) a pharmaceutically acceptable carrier or diluent.

In some embodiments, the pharmaceutical compositions disclosed herein comprises an IAP inhibitor selected from the compound of Example 1 to the compound of Example 50, or a pharmaceutically acceptable salt thereof. In one specific embodiment, the IAP inhibitor is the compound of Example 24 or a pharmaceutically acceptable salt thereof.

In some embodiments, the pharmaceutical compositions disclosed herein comprises a second inhibitor, wherein the second inhibitor is PARP inhibitor selected from talazoparib, niraparib, rucaparib, olaparib, tazemetostat, pamiparib, fluazolepali, veliparib, otaplimastat, amelparib, CK-102, 2X-121, simmiparib, SC-10914, IMP-4297, ABT-767, MP-124, RBN-2397, IDX-1197, JPI-547, HWH-340, ZYTP-1, NT-125, AST-6828, OX-401, HC-X014, CBX-11, OC-301, TSL-1502, STP06-1002, mefuparib, R-554, AZ-6102, BPI-7000, SRX-3128, JNJ-928, AZD-2461, iniparib, ONO-2231, INO-1001, INO-1003, E-7016, LT-626, JPI-283, honokiol, MK-2512, R-503, NMS-P914A, HYDAMTIQ, KR-33889, S-111, ANG-2864, PD-141703, PD-141076, PD-128763, BSI-401, A-620223, AAI-028, DR-2313, colecalciferol, and BGP-15, or a pharmaceutically acceptable salt thereof. In one specific embodiment, the PARP inhibitor is olaparib, rucaparib, niraparib, talazoparib, veliparib, pamiparib, fluazolepali, amelparib, simmiparib, mefuparib, or iniparib or a pharmaceutically acceptable salt thereof. In another specific embodiment, the PARP inhibitor is olaparib or a pharmaceutically acceptable salt thereof.

In some embodiments, the pharmaceutical compositions disclosed herein comprises a second inhibitor, wherein the second inhibitor is a MEK inhibitor selected from binimetinib, cobimetinib, trametinib, LNP-3794, HL-085, antroquinonol, E-6201, refametinib, mirdametinib, pimasertib, selumetinib, SHR-7390, CKI-27, GS-4875, ATR-001, ATR-002, ATR-006, ATR-004, ATR-005, CS-3006, FCN-159, CIP-137401, EBI-1051, SC-1-151, SRX-2626, EDV-2209, WX-554, GDC-0623, TAK-733, E-6201, RG-7167, AZD-8330, PD-184352, GSK-2091976A, AS-703988, BI-847325, JTP-70902, CZ-775, R04987655, RO5126766, RO-5068760, RDEA-436, MEK-300, AD-GL0001, SL-327, CI-1040, CInQ-03, G-573, PD184161, PD318088, PD98059, U0126, and SL327, or a pharmaceutically acceptable salt thereof. In one specific embodiment, the MEK inhibitor is trametinib or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions disclosed herein optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20^{th} edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

Provided herein are methods of treating a disease (e.g., a cancer), comprising administering to a subject in need thereof, a) an effective amount of an Inhibitors of Apoptosis Proteins (IAP) inhibitor, wherein the IAP inhibitor is represented by formula (I) or a pharmaceutically acceptable salt thereof as described above (e.g., the compound of Example 24), and b) an effective amount of a second inhibitor, wherein the second inhibitor is a poly ADP ribose polymerase (PARP) inhibitor or a mitogen-activated protein kinase kinase (MEK) inhibitor as disclosed herein, such that the disease is treated.

In the present disclosure, a compound of structural formula (I) is administered in conjunction with a second inhibitor (i.e., a PARP inhibitor or a MEK inhibitor) useful in the treatment of a disease or condition wherein inhibition of PARP or MEK provides a benefit. A compound of structural formula (I) and the second inhibitor can be administered simultaneously or sequentially to achieve the desired effect. In addition, the compound of structural formula (I) and second inhibitor can be administered from a single composition or two separate compositions.

The second inhibitor is administered in an amount to provide its desired therapeutic effect. The effective dosage range for each second inhibitor is known in the art, and the second inhibitor is administered to an individual in need thereof within such established ranges.

In certain embodiments, a combination treatment comprising administering a therapeutically effective amount of a compound of structural formula (I) and a second inhibitor produces a greater tumor response and greater clinical benefit compared to treatment with a compound of structural formula (I) or second inhibitor alone.

The compounds of structural formula (I) also can be used to achieve administration of a lower, and therefore less toxic and more tolerable, dose of a second inhibitor to produce the same tumor response/clinical benefit as the conventional dose of a second inhibitor. Also, because the compounds of structural formula (I) act at least in part by inhibiting IAP proteins, the exposure of cancer and supporting cells to therapeutically effective amounts of the present IAP protein inhibitors can be temporally linked to coincide with the attempts of cells to execute the apoptosis program in response to a second inhibitor. Thus, in some embodiments, administering the compounds of structural formula (I) in connection with a second inhibitor in certain temporal relationships provides especially efficacious therapeutic results.

A compound of structural formula (I) and the second inhibitor therefore can be administered together as a single-unit dose or separately as multi-unit doses, wherein the compound of structural formula (I) is administered before the second inhibitor or vice versa. One or more dose of the compound of structural formula (I) and/or one or more dose of the second inhibitor can be administered. The compounds of structural formula (I) therefore can be used in conjunction with one or more second inhibitor, i.e., a PARP inhibitor or a MEK inhibitor.

The diseases and conditions that can be treated in the present disclosure include, for example, cancers. A variety of cancers can be treated including, but not limited to: carcinomas, including bladder (including accelerated and metastic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma, hematopoietic tumors of myeloid lineage, including acute and chronic myelogenous leukemias, myelodysplastic syndrome, myeloid leukemia, and promyelocytic leukemia; tumors of the central and peripheral nervous system, including astrocytoma, neuroblastoma, glioma, and schwannomas; tumors of mesenchymal origin, including fibrosarcoma, rhabdomyoscarcoma, and osteosarcoma; and other tumors, including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

Additional forms of cancer treatable by the methods of the present disclosure include, for example, adult and pediatric oncology, growth of solid tumors/malignancies, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma), gastrointestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, and squamous cell cancer), thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In another embodiment, the methods disclosed herein are useful in treating T and B cell mediated autoimmune diseases; inflammatory diseases; infections; hyperproliferative diseases; AIDS; degenerative conditions; vascular diseases; and the like. In some embodiments, infections suitable for treatment with the compositions and methods of the present disclosure include, but are not limited to, infections caused by viruses, bacteria, fungi, mycoplasma, prions, and the like.

The present compositions and methods are also useful in the treatment of autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

In one embodiment, the disease can be treated by the methods of the present disclosure is a muscle structure disorder, a neuronal activation disorder, a muscle fatigue disorder, a muscle mass disorder, a beta oxidation disease, a metabolic disease, a cancer, a vascular disease, an ocular vascular disease, a muscular eye disease, or a renal disease.

In one aspect of this embodiment, the muscle structure disorder is selected from Bethlem myopathy, central core disease, congenital fiber type disproportion, distal muscular dystrophy (MD), Duchenne & Becker MD, Emery-Dreifuss MD, facioscapulohumeral MD, hyaline body myopathy, limb-girdle MD, a muscle sodium channel disorders, myotonic chondrodystrophy, myotonic dystrophy, myotubular myopathy, nemaline body disease, oculopharyngeal MD, and stress urinary incontinence.

In another aspect of the embodiment, the neuronal activation disorder is selected from amyotrophic lateral sclerosis, Charcot-Marie-Tooth disease, Guillain-Barre syndrome, Lambert-Eaton syndrome, multiple sclerosis, myasthenia gravis, nerve lesion, peripheral neuropathy, spinal muscular atrophy, tardy ulnar nerve palsy, and toxic myoneural disorder.

In another aspect of this embodiment, the muscle fatigue disorder is selected from chronic fatigue syndrome, diabetes (type I or II), glycogen storage disease, fibromyalgia, Friedreich's ataxia, intermittent claudication, lipid storage myopathy, MELAS, mucopolysaccharidosis, Pompe disease, and thyrotoxic myopathy.

In another aspect of this embodiment, the muscle mass disorder is cachexia, cartilage degeneration, cerebral palsy, compartment syndrome, critical illness myopathy, inclusion body myositis, muscular atrophy (disuse), sarcopenia, steroid myopathy, and systemic lupus erythematosus.

In another aspect of this embodiment, the beta oxidation disease is selected from systemic carnitine transporter, carnitine palmitoyltransferase (CPT) II deficiency, very long-chain acyl-CoA dehydrogenase (LCHAD or VLCAD) deficiency, trifunctional enzyme deficiency, medium-chain acyl-CoA dehydrogenase (MCAD) deficiency, short-chain acyl-CoA dehydrogenase (SCAD) deficiency, and riboflavin-responsive disorders of P-oxidation (RR-MADD).

In yet another aspect of this embodiment, the metabolic disease is selected from hyperlipidemia, dyslipidemia, hyperchlolesterolemia, hypertriglyceridemia, HDL hypocholesterolemia, LDL hypercholesterolemia and/or HLD non-cholesterolemia, VLDL hyperproteinemia, dyslipoproteinemia, apolipoprotein A-I hypoproteinemia, atherosclerosis, disease of arterial sclerosis, disease of cardiovascular systems, cerebrovascular disease, peripheral circulatory disease, metabolic syndrome, syndrome X, obesity, diabetes (type I or II), hyperglycemia, insulin resistance, impaired glucose tolerance, hyperinsulinism, diabetic complication, cardiac insufficiency, cardiac infarction, cardiomyopathy, hypertension, Non-alcoholic fatty liver disease (NAFLD), Nonalcoholic steatohepatitis (NASH), thrombus, Alzheimer disease, neurodegenerative disease, demyelinating disease, multiple sclerosis, adrenal leukodystrophy, dermatitis, psoriasis, acne, skin aging, trichosis, inflammation, arthritis, asthma, hypersensitive intestine syndrome, ulcerative colitis, Crohn's disease, and pancreatitis.

In another aspect of this embodiment, the vascular disease is selected from peripheral vascular insufficiency, peripheral vascular disease, intermittent claudication, peripheral vascular disease (PVD), peripheral artery disease (PAD), peripheral artery occlusive disease (PAOD), and peripheral obliterative arteriopathy.

In another aspect of this embodiment, the ocular vascular disease is selected from age-related macular degeneration (AMD), stargardt disease, hypertensive retinopathy, diabetic retinopathy, retinopathy, macular degeneration, retinal haemorrhage, and glaucoma.

In a further aspect of this embodiment, the muscular eye disease is selected from strabismus, progressive external ophthalmoplegia, esotropia, exotropia, a disorder of refraction and accommodation, hypermetropia, myopia, astigmatism, anisometropia, presbyopia, a disorders of accommodation, and internal ophthalmoplegia.

In a further aspect of this embodiment, the renal disease is selected from glomerulonephritis, glomerulosclerosis, nephrotic syndrome, hypertensive nephrosclerosis, acute nephritis, recurrent hematuria, persistent hematuria, chronic nephritis, rapidly progressive nephritis, acute renal failure (also known as acute kidney injury), chronic renal failure, diabetic nephropathy, and Bartter's syndrome.

In another embodiment, the disease which can be ameliorated by the methods of the present disclosure includes genetic lipodystrophy, non-alcoholic fatty liver disease (NAFLD), non-alcoholic steatohepatitis (NASH), renal ischemia/reperfusion injury (IRI), Duchenne & Becker muscular dystrophy, diabetes (type I or type II), obesity, and sarcopenia.

In another embodiment, the disease which can be ameliorated by the methods of the present disclosure includes Alpers's Disease, CPEO-Chronic progressive external ophthalmoplegia, Kearns-Sayra Syndrome (KSS), Leber Hereditary Optic Neuropathy (LHON), MELAS-Mitochondrial myopathy, encephalomyopathy, lactic acidosis, and stroke-like episodes, MERRF-Myoclonic epilepsy and ragged-red fiber disease, NARP-neurogenic muscle weakness, ataxia, and retinitis pigmentosa, Pearson Syndrome, platinum-based chemotherapy induced ototoxicity, Cockayne syndrome, xeroderma pigmentosum A, Wallerian degeneration, and HIV-induced lipodystrophy. In yet another embodiment, the disease which can be ameliorated by inhibition of PARP is acute kidney injury.

In some embodiments, the disease can be treated by the methods of the present disclosure is a cancer, selected from melanoma, colon cancer, rectal cancer, pancreatic cancer, breast cancer, lung cancer, endometrial cancer, or ovarian cancer. In one specific embodiment, the cancer is breast cancer. In one specific embodiment, the cancer is breast cancer. In one specific embodiment, the cancer is ovarian cancer. In one specific embodiment, the cancer is lung cancer. In one specific embodiment, the cancer is pancreatic cancer. In one specific embodiment, the cancer is melanoma (e.g., metastatic melanoma).

In another aspect, the methods disclosed herein are useful in treating patients with cancers that bearing BRCA1 (breast cancer type 1) and/or BRCA2 (breast cancer type 2) gene mutation. In some embodiments, a cancer that bearing BRCA1 (breast cancer type 1) and/or BRCA2 (breast cancer type 2) gene mutation is breast cancer, ovarian cancer, lung cancer, prostate cancer, pancreatic cancer, stomach cancer, colon cancer, or melanoma.

BRCA1 and BRCA2 are two tumor suppressor genes. BRCA1 and BRCA2 tumor suppressor genes have been linked to a fundamental role in the response to cellular damage through activation of specific DNA repair processes. Both BRCA1 and BRCA2 proteins are often found in stable interaction, suggesting these proteins co-function in pathways of tumor suppression. Both genes have been proposed to function in DNA repair and as transcriptional regulators. BRCA1 and BRCA2 form a complex with Rad51, a protein that has an established role in homologous recombination. Dziadkowiec et al., Menopause Rev 2016; 15(4): 215-219.

It has been shown that BRCA1 is also involved in complexing with and activation of p53. The tumor suppressor protein p53 is involved in a variety of human cancers; the normal function of p53 is to signal the occurrence of DNA damage and temporarily arrest the cell cycle to either allow repair or trigger cell death. A more detailed analysis of the effects of BRCA genes and their transcriptional functions may result in a clearer understanding of their tissue-specific actions.

PARP is a key enzyme involved in DNA repair. When a single-strand break (SSB) occurs in DNA, it is typically remedied through base excision repair via various PARP enzymes. PARP-1 is recruited/activated by SSBs, and it transfers ADP ribose moieties from cellular nicotinamide-adenine-dinucleotide (NAD*) to acceptor proteins, a process known as PAR-ylation. This eventually restores genomic integrity and normal cell function. In the absence of PARP enzymes, alternate DNA repair mechanisms still exist. An SSB can progress to a double-strand break (DSB), which can then be repaired via a precise process called homologous recombination. Wild-type BRCA1 and BRCA2 are part of the complex that permits homologous recombination. On the other hand, if DSBs cannot be repaired via homologous recombination (e.g., due to BRCA deficiencies mutations), there will be irreversible DNA damage and subsequent cell death. PARP inhibitors promote the progression of SSBs to DSBs, and can induce synthetic lethality in cells with impaired homologous recombination mechanisms. An overactivation of PARP-1 can also deplete NAD+ and lead to apoptosis.

In another aspect, the methods disclosed herein are useful in treating patients with cancers that bearing KRAS gene mutation. In some embodiments, a cancer that bearing KRAS gene mutation is mucinous adenoma, leukemia, colorectal cancer, pancreatic cancer, or lung cancer.

### Methods of Administration and Dosage Forms

In the methods of the present disclosure, a therapeutically effective amount of a compound of formula (I) and a therapeutically effective amount of a PARP inhibitor or a MEK inhibitor, typically formulated in accordance with pharmaceutical practice, are administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A compound of structural formula (I), together with a second inhibitor, can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a compound of structural formula (I) is administered in an effective amount to achieve its intended purpose. The exact formulation, route of administration, and dosage is determined by an individual physician in view of the diagnosed condition or disease. Dosage amount and interval can be adjusted individually to provide levels of a compound of structural formula (I) that is sufficient to maintain therapeutic effects.

Toxicity and therapeutic efficacy of the compounds of structural formula (I), the PARP inhibitor and the MEK inhibitor can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a compound of structural formula (I) required for use in therapy varies with the nature of the condition being treated, the length of time that activity is desired, and the age and the condition of the patient, and ultimately is determined by the attendant physician. Dosage amounts and intervals can be adjusted individually to provide plasma levels of the IAP protein inhibitor, PARP inhibitor or MEK inhibitor that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a present IAP protein inhibitor can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A compound of structural formula (I) used in a method of the present disclosure can be administered in an amount of about 0.005 to about 500 milligrams per dose, about 0.05 to about 250 milligrams per dose, or about 0.5 to about 100 milligrams per dose. For example, a compound of structural formula (I) can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing an IAP protein inhibitor of structural formula (I), or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, g/kg, 25 μg/kg, 50 μg/kg, 75 μg/kg, 100 μg/kg, 125 μg/kg, 150 μg/kg, 175 μg/kg, 200 g/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. The above dosages are exemplary of the average case, but there can be individual instances in which higher or lower dosages are merited, and such are within the scope of this invention. In practice, the physician determines the actual dosing regimen that is most suitable for an individual patient, which can vary with the age, weight, and response of the particular patient.

In certain embodiments, the IAP inhibitor is administered in an amount of about 0.005 mg/day to about 5000 mg/day, such as about 0.005, 0.05, 0.5, 5, 9, 10, 20, 30, 40, 50, 60, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day. In certain embodiments, the IAP inhibitor is administrated in an amount of about 10 mg/week to about 200 mg/week, or about 20 mg/week to about 100 mg/week, or about 20 mg/week to about 80 mg/week, such as 10, 15, 20, 25, 30, 35, 40, 50, 55, 60, 65, 70, 75, 80, 85, 90, 95, 100, 110, 120, 130, 140, 150, 150, 160, 170, 180, 190, or 200 mg/week.

In certain embodiments, the IAP inhibitor is administered in an amount of about 1 ng/kg to about 200 mg/kg, about 1 μg/kg to about 100 mg/kg, or about 1 mg/kg to about 50 mg/kg per unit dose, such as administered in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 1.5 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, and about 200 mg/kg per unit dose, and one or more (e.g., 1, 2, 3, 4, 5, 6, 7, 8, 9 or 10) unit doses are administered every day, every 2 days, every 3 days, every 4 days, every 5 days, every 6 days, or every week.

In certain embodiments, the PARP inhibitor and/or MEK inhibitor is administered in an amount of about 0.005 mg to about 5000 mg every week, every 2 weeks, every 3 weeks, or every 4 weeks, such as about 0.005, 0.05, 0.5, 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg every week, every 2 weeks, every 3 weeks, or every 4 weeks.

In certain embodiments, the PARP inhibitor and/or MEK inhibitor is administered in an amount of about 1 ng/kg to about 200 mg/kg, about 1 μg/kg to about 100 mg/kg, or about 1 mg/kg to about 50 mg/kg per unit dose, such as administered in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 1.5 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, and about 200 mg/kg per unit dose, and one or more (e.g., 1, 2, 3, 4, 5, 6, 7, 8, 9, 10, 15 or 20) unit doses are administered every week, every 2 weeks, every 3 weeks, or every 4 weeks.

In certain embodiments, the PARP inhibitor and/or MEK inhibitor is administered in an amount of about 1 mg/m² to about 200 mg/m², about 1 ag/m² to about 100 mg/m², or about 1 mg/m² to about 50 mg/kg per unit dose, such as administered in an amount of about m² per unit dose, such as administered in an amount of about 1 μg/m², about 10 μg/m², about 25 μg/m², about 50 μg/m², about 75 μg/m², about 100 μg/m², about 125 μg/m², about 150 μg/m², about 175 μg/m², about 200 μg/m², about 225 μg/m², about 250 μg/m², about 275 μg/m², about 300 μg/m², about 325 μg/m², about 350 μg/m², about 375 μg/m², about 400 μg/m², about 425 μg/m², about 450 μg/m², about 475 μg/m², about 500 μg/m², about 525 μg/m², about 550 μg/m², about 575 μg/m², about 600 μg/m², about 625 μg/m², about 650 μg/m², about 675 μg/m², about 700 μg/m², about 725 μg/m², about 750 μg/m², about 775 μg/m², about 800 μg/m², about 825 μg/m², about 850 μg/m², about 875 μg/m², about 900 μg/m², about 925 μg/m², about 950 μg/m², about 975 μg/m², about 1 mg/m², about 1 mg/m², about 1.5 mg/m², about 2.5 mg/m², about 3 mg/m², about 3.5 mg/m², about 4 mg/m², about 4.5 mg/m², about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 60 mg/m², about 70 mg/m², about 80 mg/m², about 90 mg/m², about 100 mg/m², about 125 mg/m², about 150 mg/m², about 175 mg/m², about 200 mg/m² per unit dose, and one or more (e.g., 1, 2, 3, 4, 5, 6, 7, 8, 9, 10, 15 or 20) unit doses are administered weekly.

In certain embodiments, the IAP inhibitor and PARP inhibitor and/or MEK inhibitor are administered together, concurrently, sequentially or alternately. In certain embodiments, the IAP inhibitor, PARP inhibitor and/or MEK inhibitor are administered together, concurrently, sequentially or alternately.

In certain embodiments, the IAP inhibitor is administered 1, 2, 3, 4, 5, 6, or 7 times every week. In some embodiments, the IAP is administered continuously for at least 1 week, at least 2 weeks, at least 3 weeks, at least 4 weeks, at least 5 weeks, at least 6 weeks, at least 7 weeks, or at least 8 weeks.

In certain embodiments, the PARP inhibitor and/or MEK inhibitor is administered 1, 2, 3, 4, 5, 6, or 7 times every week; 1, 2, 3, 4, 5, 6, or 7 times every 2 weeks; or 1, 2, 3, 4, 5, 6, or 7 times every 3 weeks. In some embodiments, the PARP inhibitor and/or MEK inhibitor is administered continuously for at least 1 week, at least 2 weeks, at least 3 weeks, at least 4 weeks, at least 5 weeks, at least 6 weeks, at least 7 weeks, or at least 8 weeks.

In certain embodiments, the IAP inhibitor, PARP inhibitor, or MEK inhibitor is administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, or at least 50 days, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

In certain embodiments, the IAP inhibitor, PARP inhibitor, or MEK inhibitor is administered for one or more (e.g., 1, 2, 3, 4, 5, 6, 7, 8, 9 or 10) courses of treatment, wherein each course of treatment lasts for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days or at least 50 days, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks; wherein for each course of treatment, administration is performed 1, 2, 3, 4, 5, 6, 7, 8, 9 or 10 times; and the interval between every two courses of treatment is 0, 1, 2, 3, 4, 5, 6, 7, 8, 9, 10 days, 2 weeks, 3 weeks, 4 weeks, 1 month or 2 months.

In a preferred embodiment, the amount of the IAP inhibitor, the PARP inhibitor, or the MEK inhibitor administered for each course of treatment is the same or different when administered over a plurality of courses of treatment. In some embodiments, the amount of the IAP inhibitor, PARP inhibitor, or MEK inhibitor administered in a previous course of treatment is 1-10 times, preferably 1-5 times, such as 1.5, 2, 2.5, 3, 3.5, 4, 4.5 or 5 times, of the amount administered in a subsequent course of treatment.

In certain embodiments, the IAP inhibitor, the PARP inhibitor, or the MEK inhibitor is administered by a same or different route of administration, including oral administration, intravenous injection or subcutaneous injection.

The present disclosure also provides a method of treating pancreatic cancer, comprising administering to a subject in need thereof.
a) an effective amount of an Inhibitors of Apoptosis Proteins (IAP) inhibitor as disclosed herein (e.g., a compound represented by the structural formula below:

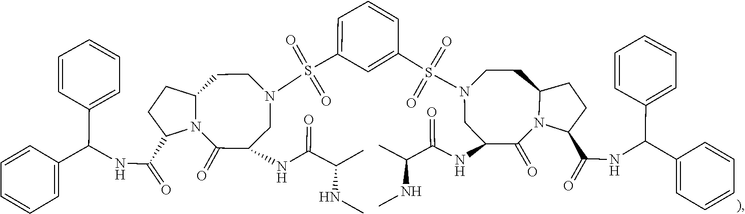

or a pharmaceutically acceptable salt thereof; wherein the effective amount of the IAP inhibitor is between 10 and 75 mg;
b) an effective amount of Abraxane; and
c) an effective amount of Gemcitabine.

In some embodiments, the method disclosed herein comprises at least one 21-day treatment cycle, wherein the IAP inhibitor is administrated on days 1, 8, and 15 of the consecutive 3-weeks of the treatment cycle. In some embodiments, the method disclosed herein comprises at least one 28-day treatment cycle, wherein the IAP inhibitor is administrated on days 1, 8, 15, and 22 of the consecutive 4-weeks of the treatment cycle.

In some embodiments, the effective amount of the IAP inhibitor is between 12-45 mg. In certain embodiments, the effective amount of the IAP inhibitor is 20 mg. In certain embodiments, the effective amount of the IAP inhibitor is 30 mg. In certain embodiments, the effective amount of the IAP inhibitor is 45 mg. In certain embodiments, the effective amount of the IAP inhibitor is 12 mg.

In some embodiments, Abraxane is administered on days 1, 8, and 15 of the consecutive 3-weeks of the treatment cycle. In some embodiments, Abraxane is administered on days 1, 8, and 15 of the consecutive 4-weeks of the treatment cycle.

In some embodiments, the effective amount of Abraxane is between 75-200 mg/m². In some embodiments, the effective amount of Abraxane is between 100-150 mg/m². In certain embodiments, the effective amount of Abraxane is 125 mg/m².

In some embodiments, Gemcitabine is administered on days 1, 8, and 15 of the consecutive 3-weeks of the treatment cycle. In some embodiments, Gemcitabine is administered on days 1, 8, and 15 of the consecutive 4-weeks of the treatment cycle.

In some embodiments, the effective amount of Gemcitabine is between 600-1500 mg/m². In some embodiments, the effective amount of Gemcitabine is between 750-1250 mg/m². In certain embodiments, the effective amount of Gemcitabine is 1000 mg/m².

In some embodiments, the IAP inhibitor is administered via an intravenous infusion. In some embodiments, Abraxane and Gemcitabine are independently administered via an intravenous infusion.

In some embodiments, Abraxane is administered first, following with Gemcitabine, and then the IAP inhibitor.

In some embodiments, the pancreatic cancer is advanced pancreatic carcinoma.

The compounds of the present invention typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing of compounds of structural formula (I).

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the compound of structural formula (I) is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a compound of structural formula (I). When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound of structural formula (I).

When a therapeutically effective amount of a compound of structural formula (I) is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

A compound of structural formula (I), a PARP inhibitor, or MEK inhibitor can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a compound of structural formula (I), and a PARP inhibitor or a MEK inhibitor can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A compound of structural formula (I), and a PARP inhibitor or a MEK inhibitor also can be formulated in rectal compositions, such as suppositories or retention enemas, e.g., containing conventional suppository bases. In addition to the formulations described previously, the compound of structural formula (I) and a PARP inhibitor or a MEK inhibitor also can be formulated as a depot preparation. Such long-acting formulations can be administered by implantation (for example, subcutaneously or intramuscularly) or by intramuscular injection. Thus, for example, the compounds of structural formula (I) and a PARP inhibitor or a MEK inhibitor can be formulated with suitable polymeric or hydrophobic materials (for example, as an emulsion in an acceptable oil) or ion exchange resins.

In particular, the compounds of structural formula (I), a PARP inhibitor or a MEK inhibitor can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The compound of structural formula (I), a PARP inhibitor or a MEK inhibitor, can also be injected parenterally, for example, intravenously, intramuscularly, subcutaneously, or intracoronarily. For parenteral administration, the IAP protein inhibitor, the PARP inhibitor or the MEK inhibitor, is best used in the form of a sterile aqueous solution which can contain other substances, for example, salts or monosaccharides, such as mannitol or glucose, to make the solution isotonic with blood.

As an additional embodiment, the present disclosure includes kits which comprise one or more compounds or compositions packaged in a manner that facilitates their use to practice methods of the invention. In one simple embodiment, the kit includes a compound or composition described herein as useful for practice of a method (e.g., a composition comprising a compound of structural formula (I) and a PARP inhibitor or a MEK inhibitor), packaged in a container, such as a sealed bottle or vessel, with a label affixed to the container or included in the kit that describes use of the compound or composition to practice the method of the invention. Preferably, the compound or composition is packaged in a unit dosage form. The kit further can include a device suitable for administering the composition according to the intended route of administration.

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th Ed., 2003).

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed compositions for guidance.

The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the present teachings may be incorporated with excipient and used in the form of ingestible tablets, buccal tablets, troches, capsules, elixirs, suspensions, syrups, wafers, and the like.

Typically for parenteral administration, solutions of a compound of the present teachings can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

The following examples are intended to be illustrative and are not intended to be limiting in any way to the scope of the disclosure.

### EXEMPLIFICATION

### Abbreviations

| | |
|---|---|
| BID | Twice a day |
| CR | Complete (tumor) regression (i.e., tumors become |
| | impalpable after treatment) |
| FSC | Forward Scatter |
| h, hr, hrs | Hour(s) |
| i.p. | Intraperitoneal injection |
| i.v. | Intravenous injection |
| Mean ± SD | Mean ± standard deviation |
| Mean ± SEM | Mean ± Standard error mean |
| mg/kg | Milligram per kilogram |
| n | Number |
| NOD | Non-obese diabetic |
| SCID | Severe combined immune deficiency |
| PK | Pharmacokinetic/Pharmacokinetics |
| PO or p.o. | Oral administration |
| PDX | Patient-derived xenograft |
| PR | Partial (tumor) regression (i.e., tumor volumes become |
| | smaller compared to the volumes before treatment) |
| QD | Once a day |
| QW | Once a week |
| Response rate | % of responsive animals in each treatment group, |
| | including CR, PR and SD |
| RTV | Relative Tumor Volume (RTV = Vₜ/V₁; V₁ and Vₜ |
| | are the average tumor volumes on the first day of |
| | treatment (day 1) and the average tumor volumes |
| | on a certain time point (day T) |
| SD | Stable disease |
| SEM | Standard error of mean |
| Synergy | Synergy score = ((A/C) × (B/C))/(AB/C); A = response |
| score/ratio | to treatment A; B = response to treatment B; |
| | C = response to vehicle control; AB = combination of |
| | treatment A and B. |
| T/C (%) | T/C (%) = (T_{RTV}/C_{RTV}) × 100%; T_{RTV} is RTV of the |
| | treatment group and C_{RTV} is RTV of the control group. |
| TV | Tumor volume |
| μL/g | Microliter per gram |
| ws | Weeks |

### Synthesis of Compounds

The preparation of IAP inhibitors of the present invention is disclosed in International Patent Publication Application No. WO2014/031487, which is incorporated herein by reference in its entirety.

### Biological Example 1

### Animals:

Balb/c nude, female, 4-6 weeks, weighing approximately 18-22 g, which were purchased from GemPharmatech Co., Ltd (Nanjing, Jiangsu).

### Generation of the Subcutaneous Pancreatic Cancer Patient-Derived Xenograft (PA1170, PA6265, PA0787, and PA1194) Model:

All of the PDX models were originally established from a surgically resected clinical sample (cancer type: pancreatic adenocarcinoma), and implanted in nude mice defined as passage 0 (P0). The next passage implanted from P0 tumor was defined as passage1 (P1), and so on during continual implantation in mice. The frozen tumor fragments were revived in NOD SCID mice, when the tumors were developed to suitable size, they were passaged to Balb/c nude mice. The P4-P7 tumors were used for the study. Whole exome sequencing and RNA sequencing revealed that BRCA2, CDKN2A, and KRAS mutation in PA1170; BRCA1, KRAS, and TP53 mutation in PA6265; CDKN2A, KRAS, TP53 mutation in PA0787; KRAS, and TP53 mutation in PA1194.

### Tumor Implantation and Animal Grouping:

Fresh tumor tissues from mice bearing established primary human cancer tissues were harvested and cut into small pieces (approximately 2-3 mm in diameter). The tumor slices of PA1170, PA6265, PA0787, and PA1194 PDX tumor, harvested from donor mice, were inoculated subcutaneously at the upper right dorsal flank into corresponding female BALB/c nude mice for tumor development. Treatments were started when the average tumor size reached approximately 150 mm³. Randomization were performed based on “Matched distribution” method/“Stratified” method using the multi-task method (StudyDirector™ software, version 3.1.399.19)/randomized block design. Each group consisted of 5 tumor-bearing mice. The testing article was administrated to the mice according to the

TABLE 1
Groups and dosing regimen for PA1170

| | | | Dose | Dosing | | Dosing |
|---|---|---|---|---|---|---|
| | | | level | Volume | | Frequency & |
| Gp | No. | Treatment | (mg/kg) | (μL/g) | ROA | Duration |
| 1 | 5 | Vehicle | — | 10 | i.v. | QW × 4 ws |
| 2 | 5 | the compound | 10 | 10 | i.v. | QW × 4 ws |
| | | of Example 24 | | | | |
| 3 | 5 | Olaparib | 50 | 10 | p.o. | BID × 4 ws |
| 4 | 5 | Trametinib | 0.3 | 10 | p.o. | QD × 4 ws |
| 5 | 2 | the compound | 10 | 10 | i.v. | QW × 4 ws |
| | | of Example 24 | | | | |
| | | Olaparib | 50 | | p.o. | BID × 4 ws |
| 6 | 2 | the compound | 10 | 10 | i.v. | QW × 4 ws |
| | | of Example 24 | | | | |
| | | Trametinib | 0.3 | | p.o. | QD × 4 ws |

Note:
a: Dosing volume will be adjusted according to individual body weight. QW: weekly; QD: daily; BID: twice daily; i.p., intraperitoneally; i.v., intravenously; p.o., orally.
b:. For combination, two-drug pack: COMPOUND OF EXAMPLE 24 will be dosed first, in 0.5 to 1 h, following with Olaparib/Trametinib/Gemcitabine; Abraxane will be dosed first, no interval, following with Gemcitabine. Three-drug pack, COMPOUND OF EXAMPLE 24 will be dosed first, in 0.5 to 1 h, following with Abraxane & Gemcitabine (no interval between each).
c: For BID, 7 h between.

TABLE 2
Groups and dosing regimen for PA0787 and PA1194

| | | | Dose | Dosing | | Dosing |
|---|---|---|---|---|---|---|
| | | | level | Volume | | Frequency & |
| Gp | No. | Treatment | (mg/kg) | (μL/g) | ROA | Duration |
| 1 | 2 | Vehicle | — | 10 | i.v. | QW × 4 ws |
| 2 | 2 | Abraxane | 30 | 10 | i.v. | QW × 4 ws |
| | | Gemcitabine | 120 | | i.p. | QW × 4 ws |
| 3 | 2 | APG-3405 | 10 | 10 | i.v. | QW × 4 ws |
| | | Abraxane | 30 | | i.v. | QW × 4 ws |
| | | Gemcitabine | 120 | | i.p. | QW × 4 ws |

Note:
a: Dosing volume was adjusted according to individual body weight.
b:. For combination, two-drug pack: the compound of Example 24 was dosed first, in 0.5 to 1 h, following with Olaparib/Trametinib/Gemcitabine; Abraxane was dosed first, no interval, following with Gemcitabine; three-drug pack: the compound of Example 24 was dosed first, in 0.5 to 1 h, following with Abraxane & Gemcitabine (no interval between each).
c: For BID, 7 h between.

### Observation:

The protocol and any amendment(s) or procedures involving the care and use of animals in this study were reviewed and approved by the Institutional Animal Care and Use Committee (IACUC) of CrownBio prior to execution. During the study, the care and use of animals were conducted in accordance with the regulations of the Association for Assessment and Accreditation of Laboratory Animal Care (AAALAC). After tumor inoculation, the animals were checked daily for morbidity and mortality. During routine monitoring, the animals were checked for any effects of tumor growth and treatments on behavior such as mobility, food and water consumption, body weight gain/loss, eye/hair matting and any other abnormalities. Mortality and observed clinical signs were recorded for individual animals in detail.

### Tumor Measurements and Endpoints:

After randomization, tumor volumes were measured twice per week in two dimensions using a caliper, and the volume was expressed in mm³ using the formula:

V=(L×W×W)/2,

where V is tumor volume, L is tumor length (the longest tumor dimension) and W is tumor width (perpendicular to L). Body weight was measured twice per week. Dosing as well as tumor and body weight measurements were conducted in a Laminar Flow Cabinet. The body weights and tumor volumes were measured by using StudyDirector™ software (version 3.1.399.19).

Relative tumor volume (RTV) was calculated using the following formula:

RTV=Vₜ/V₁,

where V₁ and Vₜ are the average tumor volumes on the first day of treatment (day 1) and the average tumor volumes on a certain time point (day t).

Synergy score was calculated using the following formula described in Clarke R. Issues in experimental design and endpoint analysis in the study of experimental cytotoxic agents in vivo in breast cancer and other models[J]. Breast Cancer Research & Treatment, 1997, 46(2-3):255-278, which is incorporated by reference in its entirety:

Synergy score=((A/C)×(B/C))/(AB/C),

where A is RTV value of drug A; B is RTV value of drug B; C is RTV value of vehicle control. AB is RTV value of combination treatment with A and B. Synergy scores: >1 represents synergistic, =1 represents additive, <1 represents antagonistic (referring to Gould S E et al. Translational value of mouse models in oncology drug development. Nature medicine. 2015 21, 431-439).

Standard NCI procedures were used to calculate tumor parameters. Percent tumor growth inhibition (% T/C) was calculated as the mean RTV of treated tumors (T) divided by the mean RTV of control tumors (C)×100%. The percentage T/C value is an indication of antitumor effectiveness: a value of T/C<42% is considered significant antitumor activity by the NCI. A T/C value <10% is considered to indicate highly significant antitumor activity, and is the level used by the NCI to justify a clinical trial if toxicity and certain other requirements are met (termed DN-2 level activity). A body weight loss nadir (mean of group) of greater than 20%, or greater than 20% of drug deaths are considered to indicate an excessively toxic dosage.

### Statistical Analysis

The antitumor activity curves of test articles were plotted with the observation time on the X-axis, and corresponding tumor volume (geometric mean) on the Y-axis. A two-tailed t-test was employed to analyze the statistical significance of any difference between the treatment group and the control group. Prism version 6 (GraphPad Software Inc., San Diego, Calif.) was used for all statistical analysis and for graphic presentation. p<0.05 is considered to be statistically significant.

### Results

### A: The Compound of Example 24 Improves Antitumor Activity of Olaparib in Pancreatic PDX Models.

The compound of Example 24 was tested in combination with olaparib in pancreatic cancer patient-derived xenograft models existing BRCA1 (PA6265) or BRCA2 (PA1170) mutation, respectively.

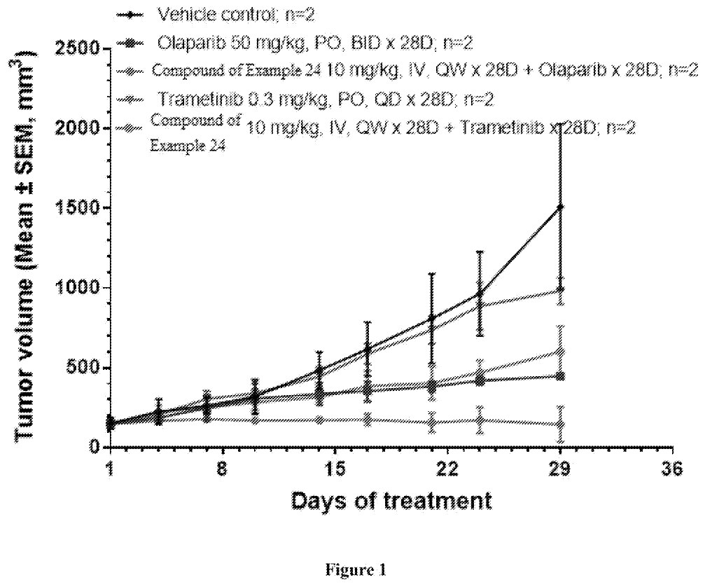

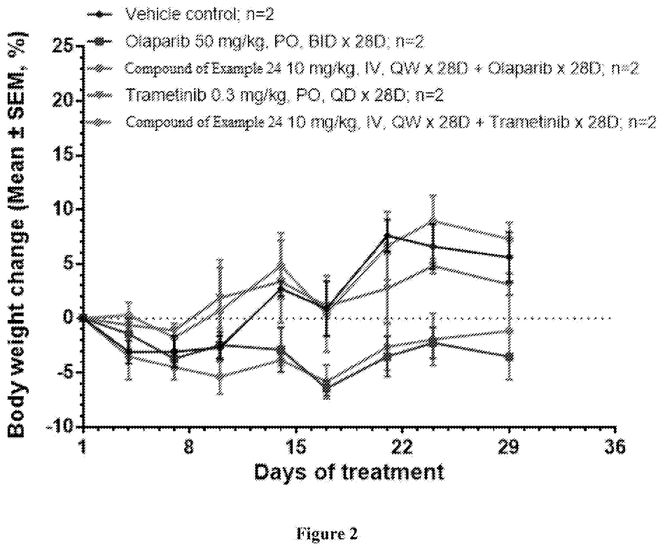

As shown in FIG. 1 and Table 3, olaparib given at 50 mg/kg p.o. BID for 4 weeks showed good activity against BRCA2 mutation bearing pancreatic cancer PDX model PA1170, with a T/C value of 32% (P<0.01) on day 28. The compound of Example 24 plus olaparib demonstrated greater antitumor activity with a T/C value of 12% (P<0.01). One out of two mice showed partial response (PR) after the combination treatment. There was no significant change in body weight during all treatments as shown in FIG. 2.

TABLE 3
RTV, T/C (%) values and synergy scores
at key time points in PA1170

| | | T/C | Synergy | |
|---|---|---|---|---|
| | | (%) @ | ratio @ | Response @ |
| Group | RTV @ D28 | D28 | D28 | D28 |
| Vehicle | 9.61 ± 1.70 | — | — | — |
| Olaparib | 3.09 ± 0.73 | 32.15 | | |
| the compound | 1.11 ± 1.27 | 11.55 | | 1/2 PR |
| of Example | | | | |
| 24 + Olaparib | | | | |
| Trametinib | 6.59 ± 0.41 | 68.57 | — | — |
| the compound | 4.10 ± 0.99 | 42.66 | — | — |
| of Example | | | | |
| 24 + Trametinib | | | | |

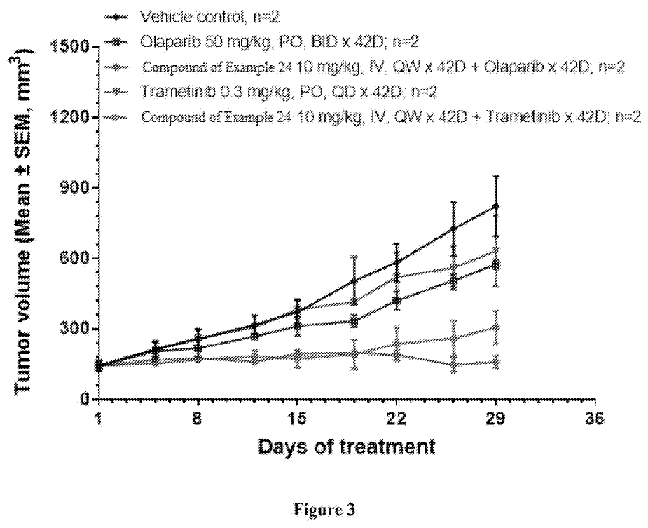

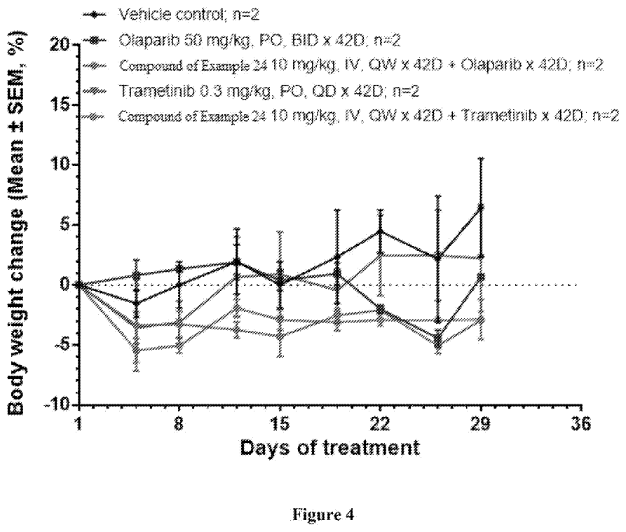

The antitumor activity of the compound of Example 24 plus olaparib in the BRCA1 mutation bearing pancreatic cancer PDX model PA6265 is showed in FIG. 3 and Table 4, olaparib at 50 mg/kg (p.o. BID for 4 weeks) had modest activities on tumor growth with a TIC value of 72% (P0.05) on day 28. However, the compound of Example 24 (10 mg/kg, i.v. QW for 4 weeks) plus olaparib showed much greater antitumor activity with a TIC value of 20% (P<0.01). Again, there was no significant change in body weight during all treatments in PA7265 as shown in FIG. 4.

TABLE 4
RTV, RTV, T/C (%) values and synergy
scores at key time points in PA6265

| | | T/C | Synergy | |
|---|---|---|---|---|
| | | (%) @ | ratio @ | Response @ |
| Group | RTV @ D29 | D29 | D29 | D29 |
| Vehicle | 5.67 ± 0.018 | — | — | — |
| Olaparib | 4.07 ± 0.51 | 71.78 | | |
| the compound | 1.14 ± 0.41 | 20.10 | | — |
| of Example | | | | |
| 24 + Olaparib | | | | |
| Trametinib | 4.31 ± 0.98 | 76.01 | — | — |
| the compound | 2.08 ± 0.50 | 36.68 | — | — |
| of Example | | | | |
| 24 + Trametinib | | | | |

Collectively, these results suggest that the combination therapy of the compound of Example 24 with olaparib achieve synergy and further enhance the antitumor effect in BRCA1 or BRCA2 mutation bearing tumor.

### B. The Compound of Example 24 Improves Antitumor Activity of Trametinib in Pancreatic PDX Models.

The compound of Example 24 was tested in combination with trametinib in KRAS mutation bearing pancreatic cancer patient-derived xenograft models PA1170 and PA6265, respectively.

As shown in FIG. 1, FIG. 3, Table 3, and Table 4, trametinib at 0.3 mg/kg p.o. QD for 4 weeks showed modest activity against pancreatic cancer PDX model PA1170 and PA6265, with a T/C value of 69% and 76% (P<0.01) on day 28, respectively. Whereas the compound of Example 24 (10 mg/kg, i.v. QW for 4 weeks) plus trametinib demonstrated greater antitumor activity with a T/C value of 43% and 37% (P<0.01) in PA 1170 and PA6265, respectively. There was no significant change in body weight during all treatments as shown in FIG. 2 and FIG. 4.

Overall, these results suggest that the combination therapy of the compound of Example 24 with trametinib achieve synergy and enhance the antitumor effect in KRAS mutation tumors.

### C. The Compound of Example 24 Improves Antitumor Activity of Abraxane Plus Gemcitabine in Pancreatic PDX Models.

The compound of Example 24 was further tested with the standard of care (SOC) of pancreatic cancer, Abraxane plus Gemcitabine, in the pancreatic cancer patient-derived xenograft model PA0787 and PA1194.

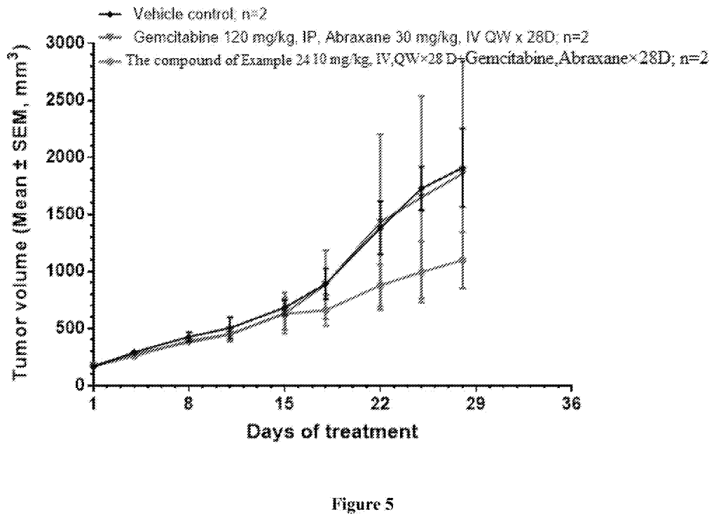

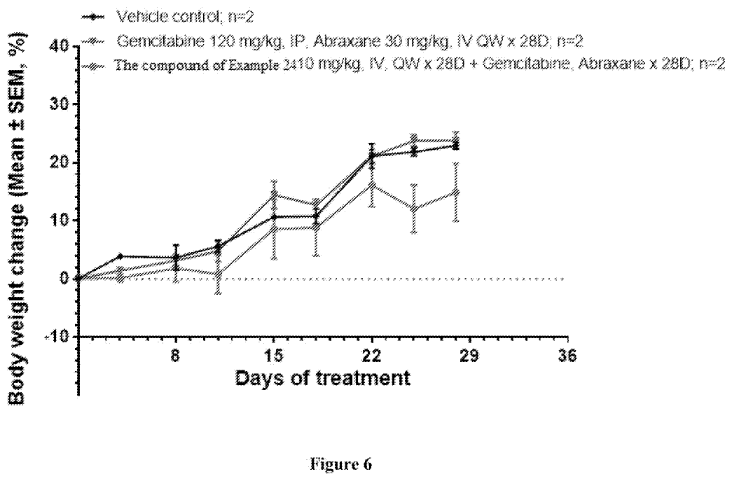

As shown in FIG. 5 and Table 5, 30 mg/kg abraxane (i.v., QW×4w) plus 120 mg/kg gemcitabine (i.p., QW×4w) had limited antitumor activities on tumor growth against pancreatic cancer PDX model PA0787 with a T/C value of 94% (P>0.05) on day 28. Combination treatments with the compound of Example 24 (10 mg/kg, i.v., QW×4w) and abraxane (30 mg/kg, i.v., QW×4w) plus gemcitabine (120 mg/kg, i.p., QW×4w) significantly inhibited tumor growth, with T/C values of 55% on day 28. No significant body weight changes were observed during all treatments as shown in FIG. 6.

TABLE 5
RTV, T/C (%) values and synergy scores
at key time points in PA0787

| | | T/C | Synergy | |
|---|---|---|---|---|
| | | (%) @ | ratio @ | Response @ |
| Group | RTV @ D28 | D28 | D28 | D28 |
| Vehicle | 11.68 ± 4.05 | — | — | — |
| Gemcitabine + | 10.97 ± 8.58 | 93.92 | | |
| Abraxane | | | | |
| the compound | 6.44 ± 2.07 | 55.14 | | — |
| of Example 24 + | | | | |
| Gemcitabine + | | | | |
| Abraxane | | | | |

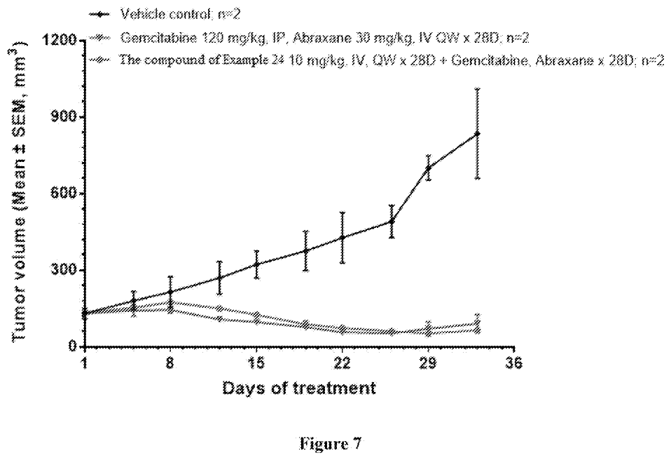

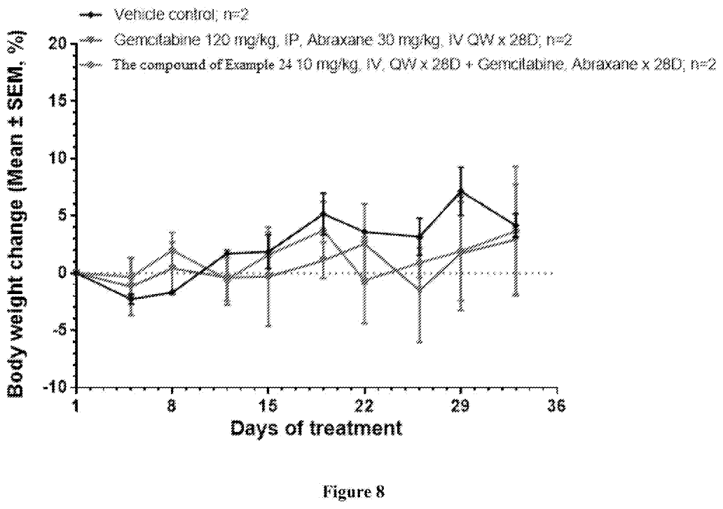

The antitumor activity of the compound of Example 24 combining with abraxane plus gemcitabine in the pancreatic cancer PDX model PA1194 is showed in FIG. 7 and Table 6. Though Abraxane (30 mg/kg, i.v., QW×4w) plus gemcitabine (120 mg/kg, i.p., QW×4w) exhibited potent antitumor activity with a T/C value of 10.3% (P<0.05) on day 33, no partial response occurred in this group. However, the addition of the compound of Example 24 (10 mg/kg, i.v. QW for 4 weeks) to the combination showed greater antitumor activity with a T/C value of 7.4% (P<0.01), and importantly, two partial response occurred in this three-drug combination treatment groups, comparing with 1 PR in abraxane plus gemcitabine treatment group. Again, no significant body weight changes were observed during all treatments in this model as shown in FIG. 8.

TABLE 6
RTV, T/C (%) values and synergy scores
at key time points in PA1194

| | | T/C | Synergy | |
|---|---|---|---|---|
| | | (%) @ | ratio @ | Response @ |
| Group | RTV @ D33 | D33 | D33 | D33 |
| Vehicle | 5.44 ± 0.66 | — | — | — |
| Gemcitabine + | 0.56 ± 0.34 | 10.29 | | 1/2 PR, 1/2 SD |
| Abraxane | | | | |
| the compound | 0.40 ± 0.12 | 7.35 | | 2/2 PR |
| of Example 24 + | | | | |
| Gemcitabine + | | | | |
| Abraxane | | | | |

Collectively, the above results suggest that the combination therapy of the compound of Example 24 with abraxane plus gemcitabine achieve synergistic antitumor effect in pancreatic cancer.

### Biological Example 2

Antitumor Activity of the Compound of Example 24 in Combination with Abraxane Plus Gemcitabine in the Treatment of Murine Pancreatic Cancer Models mPA6115 in Female C57BL/6 Mice.

### Animals:

C57BL/6, female, 6-8 weeks, weighing approximately 18-20 g, which were purchased from Shanghai Lingchang Biotechnology Co., Ltd (Shanghai, China).

### Model:

mPA6115 is a murine pancreatic cancer cell line carries a conditional mutant KRAS (KrasLSL-G12D/WT), a constitutive deletion of TP53 (p53−/−) and a Cre driven by the promoter of Pdx1 gene.

### Tumor Implantation and Animal Grouping:

Fresh tumor tissues from mice bearing established primary cancer tissues were harvested and cut into small pieces (approximately 2-3 mm in diameter). Tumor fragments, harvested from donor mice, were inoculated subcutaneously at the upper right dorsal flank into corresponding female C57BL/6 mice for tumor development. Treatments were started when the average tumor size reached approximately 150 mm³. Randomization were performed based on “Matched distribution” method/“Stratified” method using the multi-task method (StudyDirector™ software, version 3.1.399.19)/randomized block design. Each group consisted of 6 tumor-bearing mice. The testing article was administrated to the mice according to the predetermined regimen as shown in Table 7.

TABLE 7
Groups and dosing regimen for mPA6115 model

| | | | Dose | Dosing | | Dosing |
|---|---|---|---|---|---|---|
| | | | level | Volume | | Frequency & |
| Gp | No. | Treatment | (mg/kg) | (μL/g) | ROA | Duration |
| 1 | 6 | Vehicle | — | 10 | i.v. | BIW × 4 ws |
| 2 | 6 | the compound of | 6 | 10 | i.v. | BIW × 4 ws |
| | | Example 24 | | | | |
| 3 | 6 | Abraxane | 30 | 10 | i.v. | QW × 4 ws |
| | | Gemcitabine | 120 | | i.p. | QW × 4 ws |
| 4 | 6 | the compound of | 6 | 10 | i.v. | BIW × 4 ws |
| | | Example 24 | | | | |
| | | Abraxane | 30 | | i.v. | QW × 4 ws |
| | | Gemcitabine | 120 | | i.p. | QW × 4 ws |

Note:
a: Dosing volume was adjusted according to individual body weight.
b:. For combination, two-drug pack: Abraxane was dosed first, no interval, following with Gemcitabine. Three-drug pack, the compound of Example 24 was dosed first, in 0.5 to 1 h, following with Abraxane & Gemcitabine (no interval between each).

### Observation, Tumor Measurements and Endpoints, and Statistical Analysis:

Same as those described in Biological Example 1.

### Results

The Compound of Example 24 Improves Antitumor Activity of Abraxane Plus Gemcitabine in Murine Pancreatic Cancer Models mPA6115.

To confirm the synergistic antitumor effect, the antitumor activity of the compound of Example 24 in combination with Abraxane plus Gemcitabine was further examined in the murine pancreatic cancer model mPA6225.

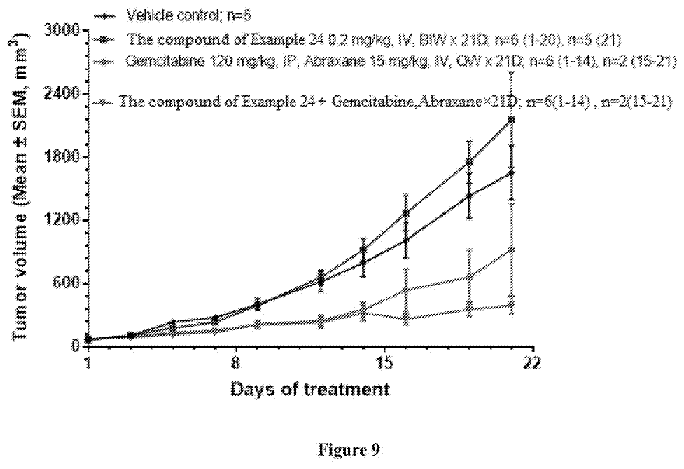

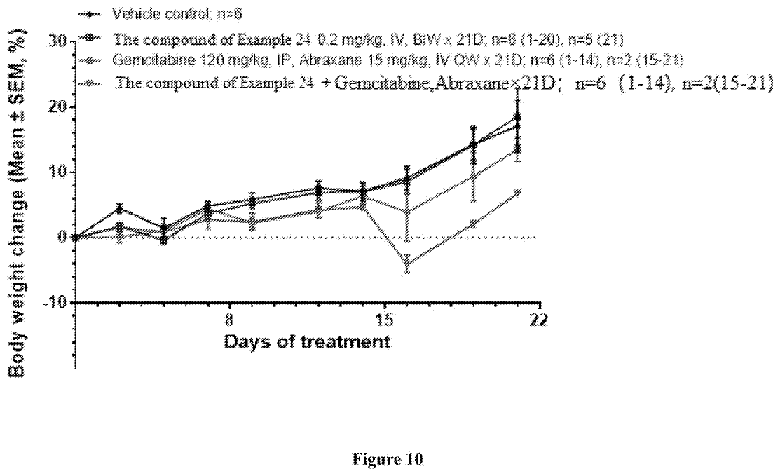

As shown in FIG. 9 and Table 8 while 15 mg/kg abraxane (i.v., QW×3w) plus 120 mg/kg gemcitabine (i.p., QW×3w) exhibited moderate antitumor activities on tumor growth with a T/C value of 52.2%, and the compound of Example 24 (0.2 mg/kg, i.v., BIW×3w) single agent showed no antitumor effect on day 21, the combination treatments with the compound of Example 24 and abraxane plus gemcitabine showed greater antitumor activity, with T/C values of 26.8% on day 21. No significant body weight changes were observed during all treatments as shown in FIG. 10. Animal death was observed in all of the drug (the compound of Example 24 or abraxane plus gemcitabine) treatment groups (Table 8), this may be due to mPA6225 model developed an extremely severe pancreatic tumor which is deadly to the mice.

TABLE 8
RTV, T/C (%) values and synergy scores
at key time points in mPA6115

| | | T/C | Synergy | |
|---|---|---|---|---|
| | | (%) @ | ratio @ | Response @ |
| Group | RTV @ D21 | D21 | D21 | D21 |
| Vehicle control | 22.71 ± 2.66 | — | — | 6/6 PD |
| the compound | 32.77 ± 10.36 | 144.28 | — | 5/6 PD, 1/6 died |
| of Example 24 | | | | |
| 0.2 mg/kg | | | | |
| Abraxane + | 11.41 ± 5.56 | 50.23 | — | 2/6 PD, 4/6 died |
| Gemcitabine | | | | |
| the compound of | 6.10 ± 0.63 | 26.84 | 2.70 | 2/6 PD, 4/6 died |
| Example 24 + | | | | |
| Abraxane + | | | | |
| Gemcitabine | | | | |

*P < 0.05, vs. vehicle control group; #P < 0.05, vs. the compound of Example 24 group; Synergy: Ratio > 1, synergistic; Ratio = 1, additive; Ratio < 1, antagonistic.

The above results confirm that the combination therapy of the compound of Example 24 with abraxane plus gemcitabine achieve synergistic antitumor effect in pancreatic cancer.

### Biological Example 3

### Compound of Example 24 Improves Antitumor Activity of Olaparib in Pancreatic PDX Models

COMPOUND OF EXAMPLE 24 was confirmed to show synergistic antitumor activity in combination with olaparib and trametinib in PA1170 pancreatic cancer patient-derived xenograft models.

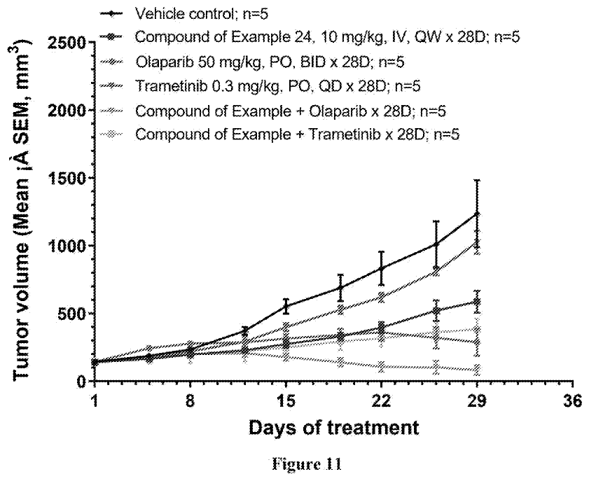

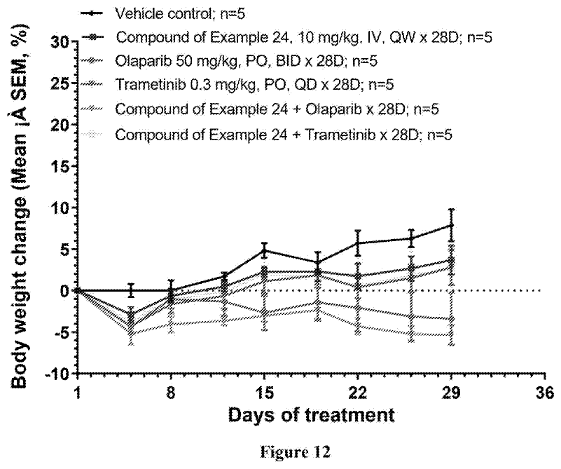

As shown in FIG. 11 and Table 9, after four weeks of dosing, IV injection of COMPOUND OF EXAMPLE 24 single agent at 10 mg/kg showed modest antitumor activity with a T/C value of 50.4% (P<0.01), and olaparib given at 50 mg/kg p.o. BID for 4 weeks showed good activity against PA1170 model and achieved a T/C value of 22.3% (P<0.01), while trametinib at 0.3 mg/kg p.o. QD for 4 weeks showed limited antitumor activity with a T/C value of 86.3% in PA1170 on day 29. In consistent with previous data, the combination of COMPOUND OF EXAMPLE 24 plus olaparib (T/C value of 7.4%, P<0.01) or trametinib (T/C value of 31.8%, P<0.01) demonstrated greater antitumor activity than single agents, achieving a synergy ratio of 1.52 and 1.37, respectively. One out of 5 mice showed completed response (CR) after the COMPOUND OF EXAMPLE 24 plus olaparib combination treatment. There was no significant change in body weight during all treatments as shown in FIG. 12.

TABLE 9
RTV, T/C (%) values and synergy scores
at key time points in PA1170

| | | T/C | Synergy | |
|---|---|---|---|---|
| | | (%) @ | ratio @ | Response @ |
| Group | RTV @ D29 | D29 | D29 | D28 |
| Vehicle | 8.51 ± 2.67 | | — | — |
| COMPOUND OF | 4.29 ± 1.83 | 50.41 | | 1/5 SD |
| EXAMPLE 24 | | | | |
| Olaparib | 1.90 ± 1.13 | 22.33 | | |
| Trametinib | 7.34 ± 1.49 | 86.25 | | |
| COMPOUND OF | 0.63 ± 0.65 | 7.40 | 1.52 | 1/5 CR, 2/5 SD |
| EXAMPLE 24 + | | | | |
| Olaparib | | | | |
| COMPOUND OF | 2.71 ± 0.47 | 31.84 | 1.37 | 3/5 SD |
| EXAMPLE 24 + | | | | |
| Trametinib | | | | |

Collectively, the results above confirmed that the combination therapy of COMPOUND OF EXAMPLE 24 with olaparib or trametinib achieve synergistic antitumor effect in BRCA1 or BRCA2 mutation bearing tumor.

### Biological Example 4

### COMPOUND OF EXAMPLE 24 Improves Antitumor Activity of Abraxane Plus Gemcitabine in Panc-1 Human Pancreatic Cancer Xenograft Model in Mice

COMPOUND OF EXAMPLE 24 was tested with the standard of care (SOC) of pancreatic cancer, Abraxane plus Gemcitabine, in the Panc-1 human pancreatic cancer xenograft model in mice.

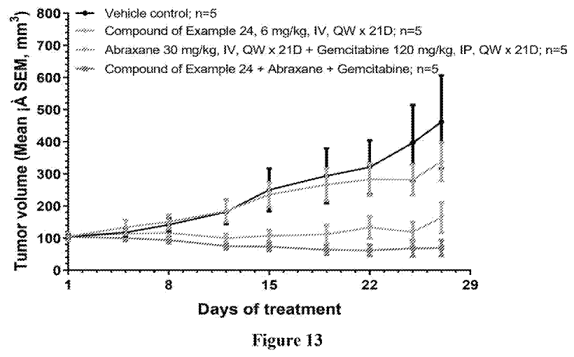

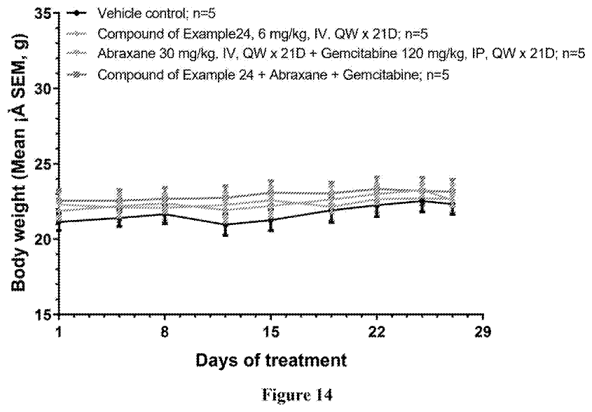

As shown in FIG. 13 and Table 10, while COMPOUND OF EXAMPLE 24 single agent had limited antitumor activities with a T/C value of 66.1% (P>0.05) on day 27, treatment with 30 mg/kg abraxane (i.v., QW×3w) plus 120 mg/kg gemcitabine (i.p., QW×3w) showed good efficact on tumor growth with a T/C value of 31.5% (P<0.05). Moreover, combination treatments with COMPOUND OF EXAMPLE 24 and abraxane plus gemcitabine significantly inhibited tumor growth (T/C values of 12.9% on day 27), and achieved a synergy ratio of 1.61 at the end of treatment. No significant body weight changes were observed during all treatments as shown in FIG. 14. The above results demonstrated that the combination therapy of COMPOUND OF EXAMPLE 24 with abraxane plus gemcitabine achieve synergistic antitumor effect in pancreatic cancer.

TABLE 10
RTV, T/C (%) values and synergy scores
at key time points in PA1170

| | | T/C | Synergy |
|---|---|---|---|
| | | (%) @ | ratio @ |
| Treatment | RTV @ D27 | D27 | D27 |
| Vehicles | 3.98 ± 1.43 | — | — |
| COMPOUND OF | 2.63 ± 0.51 | 66.08 | — |
| EXAMPLE 24 | | | |
| Abraxane + | 1.25 ± 0.41 | 31.45 | — |
| Gemcitabine | | | |
| COMPOUND OF | 0.52 ± 0.15 | 12.94 | 1.61 |
| EXAMPLE 24 + | | | |
| Abraxane + | | | |
| Gemcitabine | | | |

### Biological Example 5

Title: A Phase 1b/II Randomized, Open-Label Study of the Compound of Example 24 in Combination with Abraxane Plus Gemcitabine Inpatients with Advanced Pancreatic Carcinoma.

### Main Purpose

The primary objective of the phase 1b study is to assess the safety and tolerability of the compound of Example 24 in combination with Abraxane plus Gemcitabine in patients with advanced pancreatic carcinoma. The second objective is to determine the maximum tolerated dose (MTD) and the recommended Phase II dose (RP2D).

The primary objective of the phase II study is to assess the objective response rate (ORR) in patients with advanced pancreatic carcinoma who are resistant or refractory to the first line 5-FU chemotherapy treatment, after the treatment with the compound of Example 24 in combination with Abraxane plus Gemcitabine.

### Study Design

This is a multi-center, open-label, phase 1b dose, and phase II efficacy study.

The phase 1b study will assess the safety and tolerability of the compound of Example 24 in combination with Abraxane plus Gemcitabine in different doses, in order to determine the maximum tolerated dose (MTD) and the recommended Phase II dose (RP2D).

According to the results of the previous phase I studies, the starting dose of the present study is 20 mg, which may be adjusted to 30 mg, 45 mg, or 12 mg, based on emergent safety and tolerability data. Every four weeks is one treatment cycle.

### Investigational Product(s) (IP), Doses, Administration Methods

The compound of Example 24 will be administrated via intravenous infusion, 10 mg/vial; Abraxane will be administrated via intravenous infusion, 100 mg/vial; and Gemcitabine will be administrated via intravenous infusion, 200 mg/vial.

Abraxane: 125 mg/m² IV/30 min-35 min, the maximum infusion time does not exceed 35 min (day 1, day 8, day 15) once weekly in a 21-day-cycle, then take rest for a week, and one cycle every four weeks treatment, the administration refer to the pharmaceutical instructions; until progression or limiting toxicity;

Gemcitabine: 1000 mg/m² IV/30 min-40 min, the maximum infusion time does not exceed 40 min (day 1, day 8, day 15) once weekly in a 21-day-cycle then take rest for a week, the administration refer to the pharmaceutical instructions; and one cycle every four weeks treatment until progression or limiting toxicity;

the compound of Example 24: the dose for the II study will be based on RP2D determined in the phase 1b study; IV/30±3 min (day 1, day 8, day 15, day 22) once weekly in a 28-day-cycle. The compound of Example 24 is a freeze-dried powder for injection, 10 mg per bottle. Before use, dissolve it with sterile water for injection and then dilute with 0.9% sodium chloride. The ratio is in accordance with the pharmaceutical instructions.

Abraxane will be dosed first, no interval, following with Gemcitabine, then within 30±5 mins, following with the compound of Example 24 IV/30 min. One cycle every four weeks treatment until progression or limiting toxicity.

Abraxane: 125 mg/m² administeres as an intravenous infusion over 30-40 minutes on Days 1, 8 and 15 of each 28-day cycle, the administration refer to the pharmaceutical instructions. Until progression or limiting toxicity;

Gemcitabine: 1000 mg/m² IV/30 min-40 min, the maximum infusion time does not exceed 40 min on Days 1, 8 and 15 of each 28-day cycle, the administration refer to the pharmaceutical instructions. Until progression or limiting toxicity;

The compound of Example 24: the dose for the II study will be based on RP2D determined in the phase 1b study; IV/30±3 min (day 1, day 8, day 15, day 22) once weekly in a 28-day-cycle. The compound of Example 24 is a freeze-dried powder for injection, 10 mg per bottle. Before use, dissolve it with sterile water for injection and then dilute with 0.9% sterile saline or with 5% dextrose injection. The ratio is in accordance with the pharmaceutical instructions.

Abraxane will be dosed first, no interval, following with gemcitabine, then within 30±5 mins, following with the compound of Example 24 IV/30 min. One cycle every four weeks treatment until progression or limiting toxicity.

### REFERENCES

(1) D. W. Nicholson, Nature 2000, 407, 810-816.
(2) B. A. Ponder, Nature 2001, 411, 336-341.
(3) S. W. Lowe et al., Carcinogenesis 2000, 21, 485-495.
(4) D. Hanahan et al., Cell 2000, 100, 57-70.
(5) G. S. Salvesen et al., Nat. Rev. Mol. Cell. Biol. 2002, 3, 401-410.
(6) Q. L. Deveraux et al., Genes Dev. 1999, 13, 239-252.
(7) S. M. Srinivasula et al., Mol. Cell 2008, 30, 123-135.
(8) M. Gyrd-Hansen et al., Nat Rev Cancer, 2010, 10, 561-574.
(9) I. Tamm et al., Clin Cancer Res. 2000, 6, 1796-1803.
(10) D. Vucic et al., Clin Cancer Res. 2007, 13, 5995-6000.
(11) A. M. Hunter et al., Apoptosis 2007, 12, 1543-1568.
(12) E. C. LaCasse et al., Oncogene 2008, 27, 6252-6275.

1. A pharmaceutical composition comprising
a) an effective amount of an Inhibitors of Apoptosis Proteins (IAP) inhibitor, wherein the IAP inhibitor is represented by the formula below: 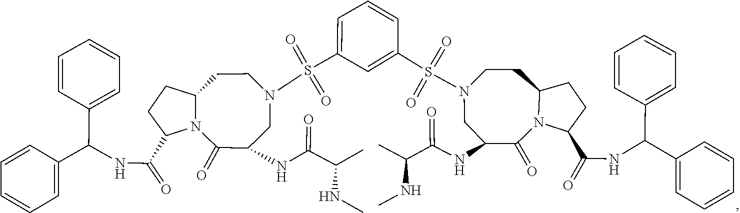
or a pharmaceutically acceptable salt thereof,
b) an effective amount of a second inhibitor, wherein the second inhibitor is a poly ADP ribose polymerase (PARP) inhibitor or a mitogen-activated protein kinase kinase (MEK) inhibitor, wherein the PARP inhibitor is olaparib, rucaparib, niraparib, talazoparib, veliparib, pamiparib, fluazolepali, amelparib, simmiparib, mefuparib, or iniparib; and wherein the MEK inhibitor is selected from binimetinib, cobimetinib, trametinib, LNP-3794, HL-085, antroquinonol, E-6201, refametinib, mirdametinib, pimasertib, selumetinib, SHR-7390, CKI-27, GS-4875, ATR-001, ATR-002, ATR-006, ATR-004, ATR-005, CS-3006, FCN-159, CIP-137401, EBI-1051, SC-1-151, SRX-2626, EDV-2209, WX-554, GDC-0623, TAK-733, E-6201, RG-7167, AZD-8330, PD-184352, GSK-2091976A, AS-703988, BI-847325, JTP-70902, CZ-775, R04987655, R05126766, RO-5068760, RDEA-436, MEK-300, AD-GL0001, SL-327, CI-1040, CInQ-03, G-573, PD184161, PD318088, PD98059, U0126, and SL327; and
c) a pharmaceutically acceptable carrier or diluent.

2-19. (canceled)

20. A method of treating a disease, comprising administering to a subject in need thereof:
a) an effective amount of an Inhibitors of Apoptosis Proteins (IAP) inhibitor, wherein the IAP inhibitor is represented by the formula below: 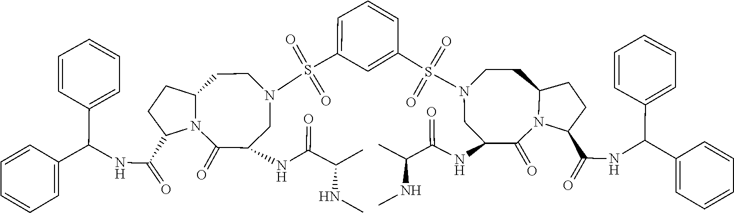
or a pharmaceutically acceptable salt thereof; and
b) an effective amount of a second inhibitor, wherein the second inhibitor is a poly ADP ribose polymerase (PARP) inhibitor or a mitogen-activated protein kinase kinase (MEK) inhibitor, wherein the PARP inhibitor is olaparib, rucaparib, niraparib, talazoparib, veliparib, pamiparib, CEP-9722, or E7016; and wherein the MEK inhibitor is selected from binimetinib, cobimetinib, trametinib, LNP-3794, HL-085, antroquinonol, E-6201, refametinib, mirdametinib, pimasertib, selumetinib, SHR-7390, CKI-27, GS-4875, ATR-001, ATR-002, ATR-006, ATR-004, ATR-005, CS-3006, FCN-159, CIP-137401, EBI-1051, SC-1-151, SRX-2626, EDV-2209, WX-554, GDC-0623, TAK-733, E-6201, RG-7167, AZD-8330, PD-184352, GSK-2091976A, AS-703988, BI-847325, JTP-70902, CZ-775, R04987655, R05126766, RO-5068760, RDEA-436, MEK-300, AD-GL0001, SL-327, CI-1040, CInQ-03, G-573, PD184161, PD318088, PD98059, U0126, and SL327; wherein the IAP inhibitor and the second inhibitor are administered concurrently or sequentially.

21-40. (canceled)

41. The method of claim 20, wherein the disease is a cancer, a T and B cell mediated autoimmune disease, and inflammatory disease; an infection, a hyperproliferative diseases, AIDS, a degenerative condition, or a vascular disease.

42. The method of claim 41, wherein the disease is selected from the group consisting of autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, and vitiligo.

43. The method of claim 20, wherein the disease is a cancer, and the cancer is melanoma, colon cancer, rectal cancer, pancreatic cancer, breast cancer, lung cancer, endometrial cancer, or ovarian cancer.

44-47. (canceled)

48. The method of claim 20, wherein the IAP inhibitor is administered in an amount of about 0.005 mg/day to about 5000 mg/day.

49. The method of claim 20, wherein the IAP inhibitor is administered in an amount of about 1 ng/kg to about 200 mg/kg, about 1 μg/kg to about 100 mg/kg, or about 1 mg/kg to about 50 mg/kg per unit dose, wherein the second inhibitor is administered in an amount of about 0.005 mg/week to about 5000 mg/week.

50. (canceled)

51. The method of claim 20, wherein the second inhibitor is administered in an amount of about 1 ng/kg to about 200 mg/kg, about 1 μg/kg to about 100 mg/kg, or about 1 mg/kg to about 50 mg/kg per unit dose.

52. A method of treating pancreatic cancer, comprising administering to a subject in need thereof:
a) an effective amount of an Inhibitors of Apoptosis Proteins (IAP) inhibitor represented by the structural formula below: 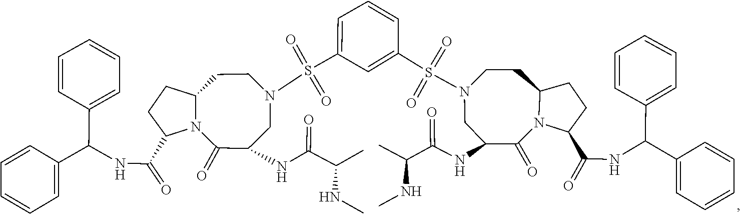
or a pharmaceutically acceptable salt thereof; wherein the effective amount of the IAP inhibitor is between 10 and 75 mg;
b) an effective amount of Abraxane; and
c) an effective amount of Gemcitabine.

53. The method of claim 52, wherein the method comprises at least one 21-day treatment cycle, wherein the IAP inhibitor is administrated on days 1, 8, and 15 of the consecutive 3-weeks of the treatment cycle.

54. The method of claim 52, wherein the method comprises at least one 28-day treatment cycle, wherein the IAP inhibitor is administrated on days 1, 8, 15, and 22 of the consecutive 4-weeks of the treatment cycle.

55. The method of claim 54, wherein the effective amount of the IAP inhibitor is between 12-45 mg.

56-59. (canceled)

60. The method of claim 52, wherein the IAP inhibitor is administered via an intravenous infusion.

61. The method of claim 55, wherein Abraxane is administered on days 1, 8, and 15 of the consecutive 3-weeks of the treatment cycle.

62. (canceled)

63. The method of claim 53, wherein the effective amount of Abraxane is between 75-200 mg/m².

64. The method of claim 54, wherein the effective amount of Abraxane is between 100-150 mg/m².

65. The method of claim 61, wherein the effective amount of Abraxane is 125 mg/m².

66. The method of claim 53, wherein Gemcitabine is administered on days 1, 8, and 15 of the consecutive 3-weeks of the treatment cycle.

67. The method of claim 64, wherein Gemcitabine is administered on days 1, 8, and 15 of the consecutive 4-weeks of the treatment cycle, wherein the effective amount of Gemcitabine is between 600-1500 mg/m².

68-72. (canceled)

73. The method of claim 52, wherein the pancreatic cancer is advanced pancreatic carcinoma.
